SEQUENCE LISTING (100 sequences)

SEQ ID NO: 1 - protein, 3 aa, Artificial Sequence
  Synthetic
RGD

SEQ ID NO: 2 - protein, 5 aa, Artificial Sequence
  Synthetic
PHSRN

SEQ ID NO: 3 - protein, 8 aa, Artificial Sequence
  Synthetic
EILDVPST

SEQ ID NO: 4 - protein, 25 aa, Artificial Sequence
  Synthetic
DELPQLVTLPHPNLHGPEILDVPST

SEQ ID NO: 5 - protein, 20 aa, Artificial Sequence
  Synthetic
GEEIQIGHIPREDVDYHLYP

SEQ ID NO: 6 - protein, 18 aa, Artificial Sequence
  Synthetic
YEKPGSPRREVVPRPRGV

SEQ ID NO: 7 - protein, 15 aa, Artificial Sequence
  Synthetic
KNNQKSEPLIGRKKT

SEQ ID NO: 8 - protein, 16 aa, Artificial Sequence
  Synthetic
YRVRVTPKEKTGPMKE

SEQ ID NO: 9 - protein, 9 aa, Artificial Sequence
  Synthetic
SPPRRARVT

SEQ ID NO: 10 - protein, 8 aa, Artificial Sequence
  Synthetic
WQPPRARI

SEQ ID NO: 11 - protein, 19 aa, Artificial Sequence
  Synthetic
VVIDASTAIDAPSNLRFLA

SEQ ID NO: 12 - protein, 8 aa, Artificial Sequence
  Synthetic
EILEVPST

SEQ ID NO: 13 - protein, 203 aa, Artificial Sequence
  Synthetic
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXRGDXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 14 - protein, 205 aa, Artificial Sequence
  Synthetic
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPHSRNXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 15 - protein, 6 aa, Artificial Sequence
  Synthetic
PHSRNC

SEQ ID NO: 16 - protein, 5 aa, Artificial Sequence
  Synthetic
HHSRN

SEQ ID NO: 17 - protein, 4 aa, Artificial Sequence
  Synthetic
HSRN

SEQ ID NO: 18 - protein, 5 aa, Artificial Sequence
  Synthetic
CRGDS

SEQ ID NO: 19 - protein, 6 aa, Artificial Sequence
  Synthetic
CCRGDS

SEQ ID NO: 20 - protein, 6 aa, Artificial Sequence
  Synthetic
PHSRNS

SEQ ID NO: 21 - protein, 7 aa, Artificial Sequence
  Synthetic
PHSRNSI

SEQ ID NO: 22 - protein, 8 aa, Artificial Sequence
  Synthetic
PHSRNSIT

SEQ ID NO: 23 - protein, 9 aa, Artificial Sequence
  Synthetic
PHSRNSITL

SEQ ID NO: 24 - protein, 10 aa, Artificial Sequence
  Synthetic
PHSRNSITLT

SEQ ID NO: 25 - protein, 11 aa, Artificial Sequence
  Synthetic
PHSRNSITLTN

SEQ ID NO: 26 - protein, 12 aa, Artificial Sequence
  Synthetic
PHSRNSITLTNL

SEQ ID NO: 27 - protein, 13 aa, Artificial Sequence
  Synthetic
PHSRNSITLTNLT

SEQ ID NO: 28 - protein, 14 aa, Artificial Sequence
  Synthetic
PHSRNSITLTNLTP

SEQ ID NO: 29 - protein, 15 aa, Artificial Sequence
  Synthetic
PHSRNSITLTNLTPG

SEQ ID NO: 30 - protein, 18 aa, Artificial Sequence
  Synthetic
PEHFSGRPREDRVPHSRN

SEQ ID NO: 31 - protein, 17 aa, Artificial Sequence
  Synthetic
EHFSGRPREDRVPHSRN

SEQ ID NO: 32 - protein, 16 aa, Artificial Sequence
  Synthetic
HFSGRPREDRVPHSRN

SEQ ID NO: 33 - protein, 15 aa, Artificial Sequence
  Synthetic
FSGRPREDRVPHSRN

SEQ ID NO: 34 - protein, 14 aa, Artificial Sequence
  Synthetic
SGRPREDRVPHSRN

SEQ ID NO: 35 - protein, 13 aa, Artificial Sequence
  Synthetic
GRPREDRVPHSRN

SEQ ID NO: 36 - protein, 12 aa, Artificial Sequence
  Synthetic
RPREDRVPHSRN

SEQ ID NO: 37 - protein, 11 aa, Artificial Sequence
  Synthetic
PREDRVPHSRN

SEQ ID NO: 38 - protein, 10 aa, Artificial Sequence
  Synthetic
REDRVPHSRN

SEQ ID NO: 39 - protein, 9 aa, Artificial Sequence
  Synthetic
EDRVPHSRN

SEQ ID NO: 40 - protein, 8 aa, Artificial Sequence
  Synthetic
DRVPHSRN

SEQ ID NO: 41 - protein, 7 aa, Artificial Sequence
  Synthetic
RVPHSRN

SEQ ID NO: 42 - protein, 6 aa, Artificial Sequence
  Synthetic
VPHSRN

SEQ ID NO: 43 - protein, 28 aa, Artificial Sequence
  Synthetic
PEHFSGRPREDRVPHSRNSITLTNLTPG

SEQ ID NO: 44 - protein, 5 aa, Artificial Sequence
  Synthetic
PPSRN

SEQ ID NO: 45 - protein, 5 aa, Artificial Sequence
  Synthetic
HHSRN

SEQ ID NO: 46 - protein, 5 aa, Artificial Sequence
  Synthetic
HPSRN

SEQ ID NO: 47 - protein, 5 aa, Artificial Sequence
  Synthetic
PHTRN

SEQ ID NO: 48 - protein, 5 aa, Artificial Sequence
  Synthetic
HHTRN

SEQ ID NO: 49 - protein, 5 aa, Artificial Sequence
  Synthetic
HPTRN

SEQ ID NO: 50 - protein, 5 aa, Artificial Sequence
  Synthetic
PHSNN

SEQ ID NO: 51 - protein, 5 aa, Artificial Sequence
  Synthetic
HHSNN

SEQ ID NO: 52 - protein, 5 aa, Artificial Sequence
  Synthetic
HPSNN

SEQ ID NO: 53 - protein, 5 aa, Artificial Sequence
  Synthetic
PHTNN

SEQ ID NO: 54 - protein, 5 aa, Artificial Sequence
  Synthetic
HHTNN

SEQ ID NO: 55 - protein, 5 aa, Artificial Sequence
  Synthetic
HPTNN

SEQ ID NO: 56 - protein, 5 aa, Artificial Sequence
  Synthetic
PHSKN

SEQ ID NO: 57 - protein, 5 aa, Artificial Sequence
  Synthetic
HHSKN

SEQ ID NO: 58 - protein, 5 aa, Artificial Sequence
  Synthetic
HPSKN

SEQ ID NO: 59 - protein, 5 aa, Artificial Sequence
  Synthetic
PHTKN

SEQ ID NO: 60 - protein, 5 aa, Artificial Sequence
  Synthetic
HHTKN

SEQ ID NO: 61 - protein, 5 aa, Artificial Sequence
  Synthetic
HPTKN

SEQ ID NO: 62 - protein, 5 aa, Artificial Sequence
  Synthetic
PHSRR

SEQ ID NO: 63 - protein, 5 aa, Artificial Sequence
  Synthetic
HHSRR

SEQ ID NO: 64 - protein, 5 aa, Artificial Sequence
  Synthetic
HPSRR

SEQ ID NO: 65 - protein, 5 aa, Artificial Sequence
  Synthetic
PHTRR

SEQ ID NO: 66 - protein, 5 aa, Artificial Sequence
  Synthetic
HHTRR

SEQ ID NO: 67 - protein, 5 aa, Artificial Sequence
  Synthetic
HPTRR

SEQ ID NO: 68 - protein, 5 aa, Artificial Sequence
  Synthetic
PHSNR

SEQ ID NO: 69 - protein, 5 aa, Artificial Sequence
  Synthetic
HHSNR

SEQ ID NO: 70 - protein, 5 aa, Artificial Sequence
  Synthetic
HPSNR

SEQ ID NO: 71 - protein, 5 aa, Artificial Sequence
  Synthetic
PHTNR

SEQ ID NO: 72 - protein, 5 aa, Artificial Sequence
  Synthetic
HHTNR

SEQ ID NO: 73 - protein, 5 aa, Artificial Sequence
  Synthetic
HPTNR

SEQ ID NO: 74 - protein, 5 aa, Artificial Sequence
  Synthetic
PHSKR

SEQ ID NO: 75 - protein, 5 aa, Artificial Sequence
  Synthetic
HHSKR

SEQ ID NO: 76 - protein, 5 aa, Artificial Sequence
  Synthetic
HPSKR

SEQ ID NO: 77 - protein, 5 aa, Artificial Sequence
  Synthetic
PHTKR

SEQ ID NO: 78 - protein, 5 aa, Artificial Sequence
  Synthetic
HHTKR

SEQ ID NO: 79 - protein, 5 aa, Artificial Sequence
  Synthetic
HPTKR

SEQ ID NO: 80 - protein, 5 aa, Artificial Sequence
  Synthetic
PHSRK

SEQ ID NO: 81 - protein, 5 aa, Artificial Sequence
  Synthetic
HHSRK

SEQ ID NO: 82 - protein, 5 aa, Artificial Sequence
  Synthetic
HPSRK

SEQ ID NO: 83 - protein, 5 aa, Artificial Sequence
  Synthetic
PHTRK

SEQ ID NO: 84 - protein, 5 aa, Artificial Sequence
  Synthetic
HHTRK

SEQ ID NO: 85 - protein, 5 aa, Artificial Sequence
  Synthetic
HPTRK

SEQ ID NO: 86 - protein, 5 aa, Artificial Sequence
  Synthetic
PHSNK

SEQ ID NO: 87 - protein, 5 aa, Artificial Sequence
  Synthetic
HHSNK

SEQ ID NO: 88 - protein, 5 aa, Artificial Sequence
  Synthetic
HPSNK

SEQ ID NO: 89 - protein, 5 aa, Artificial Sequence
  Synthetic
PHTNK

SEQ ID NO: 90 - protein, 5 aa, Artificial Sequence
  Synthetic
HHTNK

SEQ ID NO: 91 - protein, 5 aa, Artificial Sequence
  Synthetic
HPTNK

SEQ ID NO: 92 - protein, 5 aa, Artificial Sequence
  Synthetic
PHSKK

SEQ ID NO: 93 - protein, 5 aa, Artificial Sequence
  Synthetic
HHSKK

SEQ ID NO: 94 - protein, 5 aa, Artificial Sequence
  Synthetic
HPSKK

SEQ ID NO: 95 - protein, 5 aa, Artificial Sequence
  Synthetic
PHTKK

SEQ ID NO: 96 - protein, 5 aa, Artificial Sequence
  Synthetic
HHTKK

SEQ ID NO: 97 - protein, 5 aa, Artificial Sequence
  Synthetic
HPTKK

SEQ ID NO: 98 - protein, 5 aa, Artificial Sequence
  Synthetic
CRDGS

SEQ ID NO: 99 - protein, 4 aa, Artificial Sequence
  Synthetic
RGDS

SEQ ID NO: 100 - protein, 9 aa, Artificial Sequence
  Synthetic
TSHHHHHHC

(19) United States
(12) Patent Application Publication
(10) Pub. No.: US 2005/0282747 A1
(43) Pub. Date: Dec. 22, 2005

(54) Methods and compositions for wound healing

(75) Inventors: Richard A. Clark, Poquott, NY (US); Glenn Prestwich, Salt Lake City, UT (US)

Correspondence Address: MEDLEN & CARROLL, LLP, Suite 350, 101 Howard Street, San Francisco, CA, 94105, US

(73) Assignee: The Research Foundation of State University of New York at Stony Brook, Albany, NY (US)

(21) Appl. No.: 10/956,755

(22) Filed: Oct. 1, 2004

Related U.S. Application Data
(60) Provisional application No. 60/509,146, filed on Oct. 1, 2003.

(51) Int. Cl. A61K 38/18

(52) U.S. Cl. 514/12

(57) ABSTRACT

Migration-inducing peptide fragments or domains from native human fibronectin are attached through a linker to hyaluronic acid. Such agents are useful for in vivo wound healing, including but not limited to deep wounds and chronic wounds.

This invention was made with government support awarded by the National Institutes of Health. The government has certain rights in the invention.

### FIELD OF THE INVENTION

The present invention relates to methods and compositions for wound healing, and in particular, methods and compositions to promote and enhance wound healing.

### BACKGROUND

The primary goal in the treatment of wounds is to achieve wound closure. Open cutaneous wounds represent one major category of wounds and include burn wounds, neuropathic ulcers, pressure sores, venous stasis ulcers, and diabetic ulcers. Open cutaneous wounds routinely heal by a process which comprises six major components: i) inflammation, ii) fibroblast proliferation, iii) blood vessel proliferation, iv) connective tissue synthesis v) epithelialization, and vi) wound contraction. Wound healing is impaired when these components, either individually or as a whole, do not function properly. Numerous factors can affect wound healing, including malnutrition, infection, pharmacological agents (e.g., actinomycin and steroids), diabetes, and advanced age [see Hunt and Goodson in Current Surgical Diagnosis &Treatment (Way; Appleton & Lange), pp. 86-98 (1988)].

With respect to diabetes, it is known that delayed wound healing causes substantial morbidity in patients with diabetes. Diabetes mellitus is a chronic disorder of glucose metabolism and homeostasis that damages many organs. It is the eighth leading cause of death in the United States. M. Harris et al., “Prevalence of Diabetes and Impaired Glucose Tolerance and Glucose Levels in the US Population aged 20-40 Years,” Diabetes 36:523 (1987). In persons with diabetes, vascular disease, neuropathy, infections, and recurrent trauma predispose the extremities, especially the foot, to pathologic changes. These pathological changes can ultimately lead to chronic ulceration, which may necessitate amputation. In the U.S., between 300,000 and 500,000 people have diabetic ulcers.

Diabetic ulcers, however, are just one part of the chronic wound picture. It is estimated that 5.5 million people in the United States have chronic, nonhealing wounds. The care for this patient population is costly. Some chronic wounds require (on average) over $40,000 of treatment to heal. Moreover, even after such expensive treatment, there is no guarantee that the wound will not reappear.

The most commonly used conventional modality to assist in wound healing involves the use of wound dressings. Today, numerous types of dressings are routinely used, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) [Kannon and Garrett, Dermatol. Surg. 21:583-590 (1995); Davies, Burns 10:94 (1983)]. Unfortunately, certain types of wounds (e.g., diabetic ulcers, pressure sores) and the wounds of certain subjects (e.g., recipients of exogenous corticosteroids) do not heal in a timely manner (or at all) with the use of such dressings.

What is needed is a safe and effective means for enhancing the healing of wounds. The means should be able to be used without regard to the type of wound or the nature of the patient population to which the subject belongs.

### SUMMARY OF THE INVENTION

The present invention is directed at compositions and methods for enhancing the healing of wounds, especially chronic wounds (e.g., diabetic wounds, pressure sores). The compositions of the present invention are based on the discovery that certain domains or peptide fragments of fibronectin (and amino acid variants thereof) promote wound healing. The present invention contemplates the use of such peptides (or related peptide derivatives, peptide variants, protease-resistant peptides, and non-peptide mimetics) in the treatment of wounds. In particular, the present invention contemplates covalently attaching such domains, peptides, peptide derivatives, protease-resistant peptides, and non-peptide mimetics to an extracellular matrix (e.g. gelatin, collagen, hyaluronic acid, etc.). While the present invention can be successfully applied without the knowledge of any mechanism, it is believed that the extracellular matrix facilitates wound healing by providing an environment that intrinsically recruits cells to the wound site.

In a preferred embodiment, peptide fragments of fibronectin (or related peptide derivatives, protease-resistant peptides, and non-peptide mimetics) are covalently attached to a hyaluronic acid backbone (typically a derivatized hyaluronic acid) through a linker (preferably the linker is a polyethylene glycol derivative). Such constructs can be used to accelerate the healing of both acute and chronic cutaneous wounds.

It is not intended that the present invention be limited to the mode by which the compositions of the present invention are introduced to the patient. In one embodiment, the present invention contemplates topical administration of such compositions for wound healing or as a dermal filler. In another embodiment, topical administration is contemplated using solid supports (such as dressings and other matrices) and medicinal formulations (such as mixtures, suspensions and ointments). In one embodiment, the solid support comprises a biocompatible membrane. In another embodiment, the solid support comprises a wound dressing. In still another embodiment, the solid support comprises a band-aid.

In one embodiment, the present invention contemplates compositions comprising a plurality of fibronectin domains or peptide fragments. In one embodiment, the present invention contemplates a construct comprising a domain or peptide fragment of fibronectin (or related peptide derivative, peptide variant, protease-resistant peptide, or non-peptide mimetic) covalently attached to hyaluronic acid. In another embodiment, the construct comprises a domain or peptide fragment of fibronectin (or related peptide derivative, peptide variant, protease-resistant peptide, or non-peptide mimetic) covalently attached to a linker comprising polyethylene glycol (or polyethylene glycol derivative), said linker covalently attached to hyaluronic acid.

In one embodiment, the present invention contemplates a method for treating a wound, comprising a) providing: i) a construct comprising a peptide fragment of fibronectin (or related peptide derivative, peptide variant, protease-resistant peptide, or non-peptide mimetic) covalently attached to hyaluronic acid, and ii) a subject having at least one wound; and b) administering said construct to said subject under conditions such that the healing of said wound is promoted.

In one embodiment, the present invention contemplates a method for treating a wound, comprising a) providing: i) a construct comprising a peptide fragment of fibronectin (or related peptide derivative, peptide variant, protease-resistant peptide, or non-peptide mimetic) covalently attached to a linker comprising polyethylene glycol (or polyethylene glycol derivative), said linker covalently attached to hyaluronic acid, and ii) a subject having at least one wound; and b) administering said construct to said subject under conditions such that the healing of said wound is promoted.

In this embodiment, it is not intended that the present invention be limited to the type of polyethylene glycol or polyethylene glycol derivative. A variety of derivatives are known. For example, embodiments utilizing PEG-divinylsulfone, PEG-diacrylamide or PEG-diacrylate (PEGDA) are specifically contemplated. However, other derivatives can be utilized such as methyoxypoly(ethylene glycol) containing a thioimidoester reactive group, which is able to react with the lysyl epsilon-amino groups of suitable peptides or domains. S. Arpicco et al., Bioconjug. Chem. 13:757 (2002). Alternatively, amino acid type poly(ethylene glycol) is contemplated, which is prepared from poly(oxyethylene)diglycolic acid followed by introduction of a fluorenylmethyloxycarbonyl group. K. Hojo et al., Chem Pharm Bull (Tokyo) 50:1001 (2002). Still further, bis-DAP polyethylene glycol can be employed, which has diazopyruvoyl (DAP) groups attached; for example, in one embodiment the cross-linking agent N,N′-bis(3-diazopyruvoyl)-2,2′-(ethylenedioxy)bis(ethylamine) is contemplated. R. S. Givens et al., Photochem. Photobiol. 78:232 (2003). Moreover, bifunctional PEG derivatives are contemplated, such as a derivative containing both an alpha-vinyl sulfone and an omega-N-hydroxysuccinimidyl (NHS) ester group. K. Sagara et al., J Control Release 79:271 (2002).

It is not intended that the present invention be limited to the particular method by which the construct is made. In one embodiment, the method comprises a) providing a fibronectin peptide fragment, a polyethylene glycol derivative selected from the group consisting of PEG-divinylsulfone, PEG-diacrylamide and PEG-diacrylate, and hyaluronic acid; b) covalently attaching said fibronectin peptide fragment to said polyethylene glycol derivative to create a first conjugate; c) reacting said first conjugate with said hyaluronic acid to create a second conjugate.

In another embodiment, the method for linking one or more functional domains of native human fibronectin to the HA backbone comprises: a) modifying said one or more functional domains with a carboxy-terminal cysteine to create a cystein-tagged domain; b) modifying carboxylic groups of HA to contain free thiol groups so as to created thiolated HA; c) coupling said cysteine-tagged domain(s) to α, β unsaturated derivative of PEG (e.g. PEGDA) to create a first conjugate and d) crosslinking said thiolated HA to said first conjugate to create a second conjugate. In one embodiment, both steps c) and d) are done through Michael addition.

In one embodiment, the present invention also contemplates a method for treating a wound, comprising a) providing: i) a solid support comprising a peptide fragment of fibronectin (or related peptide derivative, protease-resistant peptide, or non-peptide mimetic) covalently attached to hyaluronic acid, and ii) a subject having at least one wound; and b) placing the solid support into the wound of the subject under conditions such that the healing of the wound is promoted.

It is also not intended that the present invention be limited to a particular fragment or domain of fibronectin. In one embodiment, said fibronectin-derived peptide comprises the RGD (SEQ ID NO: 1) motif, i.e. the peptide comprises the sequence Arg-Gly-Asp. In another embodiment, said peptide comprises the amino acid sequence PHSRN (SEQ ID NO: 2), i.e. the peptide comprises the sequence Pro-His-Ser-Arg-Asn. In yet another embodiment, said peptide comprises the sequence Glu-Ile-Leu-Asp-Val-Pro-Ser-Thr (SEQ ID NO: 3). In yet another embodiment, the peptide comprises the sequence Asp-Glu-Leu-Pro-Gln-Leu-Val-Thr-Leu-Pro-His-Pro-Asn-Leu-His-Gly-Pro-Glu-Ile-Leu-Asp-Val-Pro-Ser-Thr (SEQ ID NO: 4). In still another embodiment, the peptide comprises the sequence Gly-Glu-Glu-Ile-Gln-Ile-Gly-His-Ile-Pro-Arg-Glu-Asp-Val-Asp-Tyr-His-Leu-Tyr-Pro (SEQ ID NO: 5). In still another embodiment, the peptide comprises the sequence Tyr-Glu-Lys-Pro-Gly-Ser-Pro-Arg-Arg-Glu-Val-Val-Pro-Arg-Pro-Arg-Gly-Val (SEQ ID NO: 6). In still another embodiment, the peptide comprises the sequence Lys-Asn-Asn-Gln-Lys-Ser-Glu-Pro-Leu-Ile-Gly-Arg-Lys-Lys-Thr (SEQ ID NO: 7). In yet another embodiment, the peptide comprises the sequence Tyr-Arg-Val-Arg-Val-Thr-Pro-Lys-Glu-Lys-Thr-Gly-Pro-Met-Lys-Glu (SEQ ID NO: 8). In still another embodiment, the peptide comprises the sequence Ser-Pro-Pro-Arg-Arg-Ala-Arg-Val-Thr (SEQ ID NO: 9). In yet another embodiment, the peptide comprises the sequence Trp-Gln-Pro-Pro-Arg-Ala-Arg-Ile (SEQ ID NO: 10). In a further embodiment, the peptide comprises the sequence Val-Val-Ile-Asp-Ala-Ser-Thr-Ala-Ile-Asp-Ala-Pro-Ser-Asn-Leu-Arg-Phe-Leu-Ala (SEQ ID NO: 11). In yet an additional embodiment, the peptide comprises the sequence Glu-Ile-Leu-Glu-Val-Pro-Ser-Thr (SEQ ID NO: 12).

In one embodiment, the present invention contemplates a composition comprising a peptide, said peptide comprising at least three (and more preferably, at least five) contiguous amino acids from native human fibronectin (whether or not it contains other amino acids), covalently attached to a linker, said linker selected from the group consisting of polyethylene glycol and polyethylene glycol derivatives, said linker covalently attached to hyaluronic acid (preferably derivatized hyaluronic acid). While it is not intended that the present invention be limited to a particular PEG linker, in this embodiment, a preferred linker comprises a polyethylene glycol derivative selected from the group consisting of PEG-divinylsulfone, PEG-diacrylamide and PEG-diacrylate. It is also not intended that the present invention be limited to the particular peptide. In one embodiment, the present invention contemplates said peptide has the general formula: X₁RGDX₂ wherein X₁ represents between 0 and 100 additional amino acids, and X₂ of between 0 and 100. In another embodiment, said peptide has the general formula: X₁PHSRNX₂ wherein X₁ represents between 0 and 100 additional amino acids, and X₂ of between 0 and 100. Indeed, each of the peptides listed above (SEQ ID NOS 1, 2, 3, 4, 5, 6, 7, 8, 9, 10, 11 and 12) are contemplated in this general formula (wherein the peptide may or may not be flanked by additional amino acids). In a particular embodiment, said peptide sequence is flanked by one or more terminal cysteines.

The present invention also contemplates methods of treatment. In one embodiment, the present invention contemplates a method for treating a wound, comprising a) providing: i) a composition comprising a peptide, said peptide comprising at least three (and more preferably, at least five) contiguous amino acids from native human fibronectin (whether or not it contains other amino acids), covalently attached to a linker, said linker selected from the group consisting of polyethylene glycol and polyethylene glycol derivatives, said linker covalently attached to hyaluronic acid (preferably derivatized hyaluronic acid) and ii) a subject having at least one wound; and b) administering said composition to said subject under conditions such that the healing of said wound is promoted. Again, it is not intended that the method be limited by the linker type. A variety of linkers are contemplated, including but not limited to a polyethylene glycol derivative selected from the group consisting of PEG-divinylsulfone, PEG-diacrylamide and PEG-diacrylate. Again, a variety of peptides are contemplated, including those listed above (SEQ ID NOS 1, 2, 3, 4, 5, 6, 7, 8, 9, 10, 11 and 12), as well as peptide fragments of fibronectin of the general formula: X₁-peptide-X₂ wherein X₁ represents between 0 and 100 additional amino acids, and X₂ of between 0 and 100. Again, peptides with one or more terminal cysteine residues are contemplated. Most importantly, all types of subjects are contemplated, including but not limited to humans that are diabetic, immunocompromised, aged (e.g. from nursing homes), bed-ridden, malnourished. While the compositions and methods of the present invention are applicable to acute wounds (e.g. surgical wounds and burns), they are particularly applicable to chronic wounds, such as venous ulcers and the like.

The present invention is not limited to the use of only short peptides or single peptides. Rather, the present invention contemplates embodiments wherein domains of fibronectin and multiple domains of fibronectin (both contiguous and non-continguous with respect to one another) are employed. In one embodiment, the present invention contemplates a composition, comprising at least two domains from native human fibronectin, covalently attached to a linker, said linker selected from the group consisting of polyethylene glycol and polyethylene glycol derivatives, said linker covalently attached to hyaluronic acid (preferably derivatized hyaluronic acid). As with earlier described embodiments, it is not intended that the composition be limited to the linker type. In a preferred embodiment, the linker comprises a polyethylene glycol derivative, such as one selected from the group consisting of PEG-divinylsulfone, PEG-diacrylamide and PEG-diacrylate. Importantly, when the domains are attached, they also can be either contiguous or non-contiguous. In a preferred embodiment, three domains from native human fibronectin are covalently attached to said linker. In a particularly preferred embodiment, said three domains are the RGD cell binding site, a heparin II binding site and a binding site for the integrin. For ease of attachment, in one embodiment said three domains are modified by the addition of cysteine prior to covalently attaching said domains to said linker. Importantly, it is not intended that the present invention be limited to particular domains. In one embodiment, at least one of said domains comprises an amino acid sequence selected from the group consisting of SEQ ID NOS 1, 2, 3, 4, 5, 6, 7, 8, 9, 10, 11 and 12. The present invention contemplates methods for treating a wound with the composition comprising domains. In one embodiment, the method comprises a) providing: i) the composition (comprising domains attached in the manner described above) and ii) a subject having at least one wound; and b) administering said composition to said subject under conditions such that the healing of said wound is promoted. Again, there is no limitation as to the subject or wound type. In one embodiment, the subject is diabetic. In a particular embodiment, the wound is a burn; in another embodiment, it is a chronic wound. In still another, it is surgical wound.

The present invention contemplates synthesis and/or manufacturing methods. In one embodiment, the method, comprises a) providing a fibronectin peptide fragment, a polyethylene glycol derivative selected from the group consisting of PEG-divinylsulfone, PEG-diacrylamide and PEG-diacrylate, and hyaluronic acid; b) covalently attaching said fibronectin peptide fragment to said polyethylene glycol derivative to create a first conjugate; c) reacting said first conjugate with said hyaluronic acid to create a second conjugate. In another embodiment, the method, comprises a) providing at least two domains of native human fibronectin, a polyethylene glycol derivative selected from the group consisting of PEG-divinylsulfone, PEG-diacrylamide and PEG-diacrylate, and hyaluronic acid; b) covalently attaching said domains to said polyethylene glycol derivative to create a first conjugate; c) reacting said first conjugate with said hyaluronic acid to create a second conjugate.

It is not intended that the present invention be limited by the length of the peptide. Additional amino acids (whether from native human fibronectin or not) can be added to either end of the peptides (e.g. additional amino acids can be added to a sequence selected from the group consisting of SEQ ID NOS 1-12). In one embodiment, one or more cysteines is added to facilitate conjugation to other compounds, including linkers. By way of example, the synthetic peptide NH₂— PHSRNC can be prepared commercially (e.g. Multiple Peptide Systems, San Diego, Calif.). Of course, the present invention is not limited to merely the addition of one type of amino acid or the number of amino acids. In one embodiment, said peptide is between three and five hundred amino acids in length, more preferably between five and one hundred amino acids in length, still more preferably between five and twenty amino acids in length. For example, in the embodiment where said peptide comprises the amino acids PHSRN (SEQ ID NO: 2), it is contemplated that additional amino acids may be added to the amino terminus. In another embodiment, said peptide comprises the amino acids PHSRN (SEQ ID NO: 2) and additional amino acids added to the carboxy terminus. In yet another embodiment, said peptides comprises the amino acids PHSRN (SEQ ID NO: 2) and additional amino acids added to both the amino and carboxy termini.

In one embodiment, the present invention contemplates constructs comprising peptides that are (at least partially) protease resistant. In one embodiment, such protease-resistant peptides are peptides comprising protecting groups. In another embodiment, endoprotease-resistance is achieved using peptides which comprise at least one D-amino acid.

It is also not intended that the present invention be limited by the number of different domains employed. In one embodiment, three functional domains are employed. For example, the RGD cell binding site (FNIII₍₈₋₁₁₎), the heparin II binding site (FNIII₍₁₂₋₁₄₎ or FNIII₍₁₂₋₁₅₎) and binding sites for integrin (IIICS) (see FIG. 1) are employed for optimal adult human fibroblast migration. In another embodiment, only the RGD cell-binding domain is employed for optimal neonatal human fibroblast migration.

In certain embodiments, additional components are added to promote cell migration and wound healing, including cytokines and growth factors. It is not intended that the present invention be limited to a particular cytokine or growth factor. A variety of cytokines and growth factors are known (see Table 1). Such cytokines and growth factors can be used alone or in combination with other cytokines and growth factors. Such cytokines and growth factors can be administered together with the various extracellular matrices. In some embodiments, such cytokines and growth factors are not associated with the extracellular matrix or constructs, but are simply administered along with these components. By contrast, in some embodiments, the cytokine or growth factor(s) is reversibly associated with the matrix (e.g. adsorbed on the matrix, imbedded, coating the matrix, etc.). In other embodiments, the cytokine or growth factor is irreversibly associated, e.g. covalently bound to part of the construct (e.g. covalently bound to hyaluronic acid, covalently bound to a fibronectin fragment, or covalently bound to a linker). In a preferred embodiment, the growth factor PDGF is employed together with the various embodiments of the construct described above.

### DEFINITIONS

To facilitate understanding of the invention set forth in the disclosure that follows, a number of terms are defined below.

As used herein, “hyaluronic acid” is intended to include the various forms of hyaluronic acid (HA) known in the art, including hyaluronan. These various forms include HA chemically modified (such as by crosslinking) to vary its resorption capacity

TABLE 1

| Name | Abbr. | Type | Specific Name |
|---|---|---|---|
| Interferons | IFN | alpha | Leukocyte Interferon |
| | | beta | Fibroblast Interferon |
| | | gamma | Macrophage Activation |
| | | | Factor |
| Interleukins | IL-1 | 1 alpha | Endogenous Pyrogen |
| | | 1 beta | Lymphocyte-Activating |
| | | | Factor |
| | | 1 ra | IL-1 Receptor Antagonist |
| | IL-2 | | T-cell Growth Factor |
| | IL-3 | | Mast Cell Growth Factor |
| | IL-4 | | B-cell Growth Factor |
| | IL-5 | | Eosinophil Differentiation |
| | | | Factor |
| | IL-6 | | Hybridoma Growth Factor |
| | IL-7 | | Lymphopoietin |
| | IL-8 | | Granulocyte Chemotactic |
| | | | Protein |
| | IL-9 | | Megakaryoblast Growth |
| | | | Factor |
| | IL-10 | | Cytokine Synthesis |
| | | | Inhibitor Factor |
| | IL-11 | | Stromal Cell-Derived |
| | | | Cytokine |
| | IL-12 | | Natural Killer Cell |
| | | | Stimulatory Factor |
| Tumor Necrosis | TNF | alpha | Cachectin |
| Factors | | beta | Lymphotoxin |
| Colony Stimulating | CSF | GM-CSF | Granulocyte-macrophage |
| Factors | | | Colony-Stimulating Factor |
| | | Mp-CSF | Macrophage Growth Factor |
| | | G-CSF | Granulocyte Colony- |
| | | | stimulating Factor |
| | | EPO | Erythropoietin |
| Transforming | TGF | beta 1 | Cartilage-inducing |
| Growth Factor | | | Factor |
| | | beta 2 | Epstein-Barr Virus- |
| | | | inducing Factor |
| | | beta 3 | Tissue-derived Growth |
| | | | Factor |
| Other Growth | LIF | | Leukemia Inhibitory |
| Factors | | | Factor |
| | MIF | | Macrophage Migration- |
| | | | inhibiting Factor |
| | MCP | | Monocyte Chemoattractant |
| | | | Protein |
| | EGF | | Epidermal Growth Factor |
| | PDGF | | Platelet-derived Growth |
| | | | Factor |
| | FGF | alpha | Acidic Fibroblast Growth |
| | | | Factor |
| | | beta | Basic Fibroblast Growth |
| | | | Factor |
| | ILGF | | Insulin-like Growth Factor |
| | NGF | | Nerve Growth Factor |
| | BCGF | | B-cell growth factor |

and/or its ability to be degraded. HA formulations will be resorbable in a matter of months, and more preferably in a matter of weeks, and most preferably within a few days to one week.

The term “wound” refers broadly to injuries to the skin and subcutaneous tissue initiated in different ways (e.g., pressure sores from extended bed rest and wounds induced by trauma) and with varying characteristics. Of course, wounds can also be made surgically or by disease (e.g. cancer). Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds. The present invention contemplates treating all wound types, including deep wounds and chronic wounds.

The term “chronic wound” refers to a wound that has not healed within 30 days.

In one embodiment, the method of the present invention contemplates positioning the composition (comprising attached peptides or domains) in the wound (whether alone or as part of a solid support). The phrase “positioning in the wound” and “positioning the solid support in or on the wound” is intended to mean contacting (including covering) some part of the wound with the composition or solid support.

The phrases “promote wound healing,” “enhance wound healing,” and the like refer to either the induction of the formation of granulation tissue of wound contraction and/or the induction of epithelialization (i.e., the generation of new cells in the epithelium). Wound healing is conveniently measured by decreasing wound area. It is not intended that phrases such as “promote wound healing” or “enhance wound healing” require a quantitative comparison with controls. In the case of treatment of a chronic wound, it is sufficient that evidence of wound healing begin after treatment.

The term “subject” refers to both humans and animals.

The term “solid support” refers broadly to any support, including, but not limited to, microcarrier beads, gels, Band-Aids™ and dressings.

The term “dressing” refers broadly to any material applied to a wound for protection, absorbance, drainage, etc. Thus, adsorbent and absorbent materials are specifically contemplated as a solid support. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) [Kannon and Garrett, Dermatol. Surg. 21:583-590 (1995); Davies, Burns 10:94 (1983)]. The present invention specifically contemplates the use of dressings impregnated with the wound healing promoting and enhancing compounds of the present invention.

The term “biocompatible” means that there is minimal (i.e., no significant difference is seen compared to a control), if any, effect on the surroundings. For example, in some embodiments of the present invention, the dressing comprises a biocompatible membrane.

The term “mimetic” is meant to include 1) peptides modified to be resistant to proteases (i.e. to increase half-life); 2) peptides modified to replace one or more amino acids (or portions thereof) with one or more non-amino acid moieties, and 3) small molecules containing no amino acids, but mimicking the activity (e.g. binding) of a peptide. The mimetic containing no amino acids is a “non-peptide mimetic.”

The term “peptide derivative” refers to a mimetic compound comprising amino acids linked together through an imino group (—NH—) where at least one linkage lacks aspects characteristic of peptide bonds. For example, one or more linkages in a peptide derivative may lack a peptide bond because the —CO— group of the peptide bond is replaced with a CH₂ group or the like.

The term “peptide variant” refers to an amino acid sequence that contains one or more amino acid substitutions, deletions or additions relative to a native fibronectin amino acid sequence. Substitutions include the case where one or more amino acids are substituted with modified amino acids. By way of example of substitution with a modified amino acid, the present invention contemplates an embodiment wherein the modified amino acid has a different side chain (e.g. glycine, for example, can be replaced with an N-alkylated glycine such as N-isobutylglycine). By way of example of a substitution with an unmodified amino acid (and there are numerous examples provided herein), a variant of the native PHSRN (SEQ ID NO: 2) amino acid sequence is HHSRN (containing a substitution). Another variant is HSRN (illustrating a deletion). It is preferred that, for every five amino acids of a native fibronectin sequence, only one change is made in order to preserve function.

“Protecting groups” are those groups which prevent undesirable reactions (such as proteolysis) involving unprotected functional groups. In one embodiment, the present invention contemplates that the protecting group is an acyl or an amide. In one embodiment, the acyl is acetate. In another embodiment, the protecting group is a benzyl group. In another embodiment, the protecting group is a benzoyl group. The present invention also contemplates combinations of such protecting groups.

The term “Band-Aid™”, is meant to indicate a relatively small adhesive strip comprising and adsorbent pad (such as a gauze pad) for covering minor wounds.

### DESCRIPTION OF THE DRAWINGS

FIG. 1 is a schematic to illustrate the general structure of fibronectin, showing the number and relative positions of the basic functional domains.

FIG. 2 is a schematic showing the conjugation of RGD peptides (with either one or two cysteine residue added) to an excess of PEGDA to give a mixture of unreacted homobifunctional PEGDA plus monofunction peptide-PEG acrylate.

FIG. 3 shows the reaction kinetics of conjugate addition of RGD peptides with cysteine residue(s) to PEGDA.

FIG. 4 shows the influence of RGD peptide concentration and structure on fibroblast spreading on the hydrogel surface by comparing the percent of rounded cells (FIG. 4A) and the percent of cells spreading (FIG. 4B).

FIG. 5 is a simple schematic illustrating one (non-limiting) model of how two spacers may retard the peptide flexibility and make them less available for cell recognition.

FIG. 6 is a bar graph showing the impact on spreading as the PEG spacer molecular weight increased.

FIG. 7 is a bar graph showing the cell proliferation of fibroblasts on the hydrogel surface (PEGDA 3400) comprising different peptides, as compared to proliferation in a standard cell culture on a polystyrene surface (ps).

FIG. 8 is a bar graph showing the extent of proliferation of NIH 3T3 fibroblasts on the CRGDS coupled hydrogel surface using PEGDA 3400 (CRGDS concentration in bulk 0.268 mM), with cell culture polystyrene (ps) as the control.

FIG. 9 is a schematic of recombinant domains of native human fibronectin useful in conjunction with certain embodiments of the present invention.

FIG. 10 is a schematic showing the chemical reactions for derivatizing hyaluronic acid.

FIG. 11 schematically contrasts the susceptibility of a PEGDA and a PEGDVS cross-linker to hydrolytic degradation.

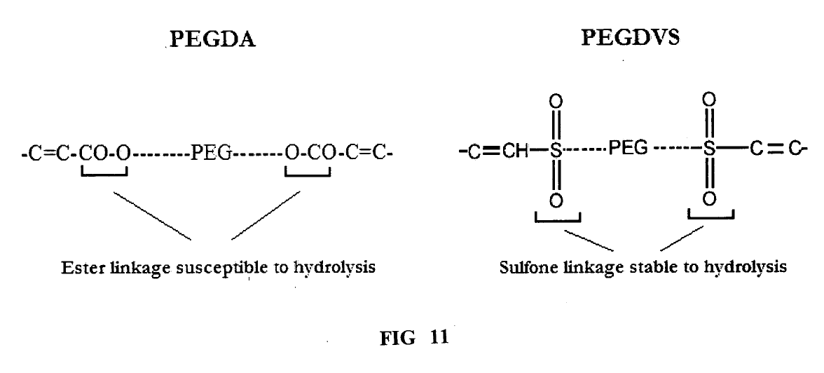

FIG. 12 is a bar graph showing the effect of rapid (PEGDA hydrogel) and slow (PEGDVS hydrogel) degradation on the tendency of fibroblast cell bodies to round up or to spread.

FIG. 13 is a schematic representation of the agarose droplet migration assay known in the art.

FIG. 14 represents, in pixels², the results of an analysis of photomicrographs of fibroblasts that have out-migrated from agarose droplets seeded with cells and deposited on hydrogel surfaces 18 hours earlier.

FIG. 15 is a diagrammatically depicted classification of engineered extracellular matrix (engECM) constructs of the invention.

FIG. 16 is a bar graph showing the effects of several engECM constructs on re-epithelalization of wounds in a porcine skin-wound model.

### GENERAL DESCRIPTION OF THE INVENTION

The present invention contemplates methods and compositions that stimulate the invasion of the wound by the cells which synthesize the growth factors and cytokines active in stimulating wound repair, especially monocytes, macrophages, keratinocytes, and fibroblasts. This strategy allows the cells in their normal in vivo setting to secrete the active factors. In one embodiment, peptide fragments of fibronectin are covalently attached to hyaluronic acid.

Hyaluronan (HA), a major constituent of extracellular matrix (ECM), is a non-sulfated glycoaminoglycan (GAG) consisting of repeating disaccharide units (α-1,4-D-glucuronic acid and β-1,3-N-acetyl-D-glucosamine). This polyanionic GAG has excellent biocompatibility, biodegradability, and also many important biological functions such as stabilizing and organizing the ECM, regulating cell adhesion and motility, and mediating cell proliferation and differentiation. As a consequence, HA and its derivatives have become widely used in clinical medicine.

HA-based hydrogels have significant potential in tissue regeneration by combining the HA biological functions with its desirable physiochemical properties. For example, HA hydrogels have high water content and physical characteristics similar to soft tissues, including high permeability for oxygen, nutrients, and other water-soluble metabolites. However, the development of HA-based hydrogels for cell growth and tissue remodeling has been impeded by poor cell attachment, since protein deposition and cell attachment are thermodynamically unfavorable due to repulsion between the net negative charges on cell surface and the polyanionic GAG surface.

To avoid attachment problems, the present invention contemplates the use of HA-based materials for tissue engineering wherein the physical and chemical modifiers are incorporated or attached to produce a porous scaffold conducive to initial cell attachment, spreading, migration, thus regulating cell function and subsequent tissue formation both in vitro and in vivo.

Disulfide synthetic mimics of the extracellular matrix have been prepared in which thiol-modified hyaluronan and thiol-modified gelatin were co-crosslinked. Both hydrogels and sponges were prepared, and both were successfully employed for cell culture ex vivo and growth of new tissues in vivo. However, cross-linking of these materials occurs only very slowly, and virtually not at all in vivo. Although the materials could be seeded with cells and then cross-linked in air, surgical implantation of the seeded scaffold was then required. Therefore an alternative approach has been developed that permits gelation in vivo of a biocompatible material which, because it is a hydrogel, is injectable. We have applied the technology to tissue repair and regeneration in a number of embodiments using an in situ crosslinkable hydrogel based on thiolated HA.

Cells can live and proliferate on and in the compositions of the present invention. For example, murine L-929 fibroblasts were encapsulated in situ in the hydrogel under physiological conditions due to oxidation of thiols to disulfide by oxygen and they remained viable and proliferated following the culture in vitro.

A variety of attachment chemistries can be used. In one embodiment, the present invention contemplates a hydrogel based on the conjugate, or Michael-addition crosslinking, between thiolated HA and poly(ethylene glycol) diacrylate (PEGDA). This embodiment is injectable for in vivo tissue engineering applications. Preliminary results showed that T31 human tracheal scar fibroblasts proliferated in this hydrogel ten-fold during 4 weeks in vitro culture, and the maintained the same phenotype during this time. Furthermore, immunohistochemistry showed fibronectin-positive staining of the resulting fibrous tissue growing in the implants of this cell-loaded hydrogel in nude mice, demonstrating that human fibroblasts proliferated and functioned in vivo.

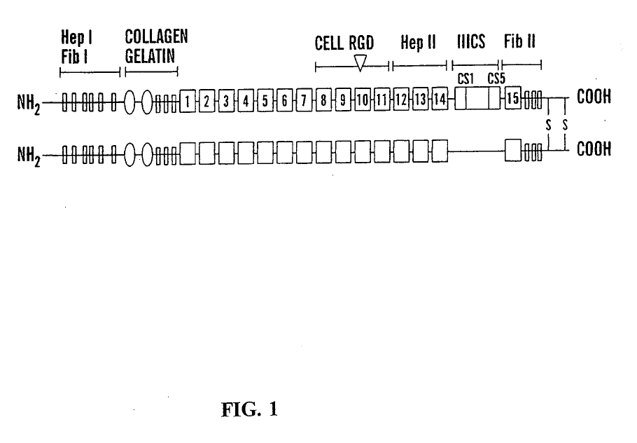

It is not intended that the present invention be limited to a particular fibronectin peptide fragment. In one embodiment, the peptide fragment comprises one or more functional domains of native human fibronectin. Although encoded by only a single gene, fibronectin exists in a number of variant forms that differ in sequence at three general regions of alternative splicing of its precursor mRNA. Some of this alternative splicing involves cell adhesion sequences, thereby providing a post-transcriptional mechanism for potentially regulating cell interaction. Nevertheless, all fibronectin molecules appear to consist of the same basic functional domains. As shown in FIG. 1, these domains include two heparin binding domains, Hep I and Hep II; two fibrin binding domains, Fib I and Fib II; a collagen or gelatin binding domain; a cell-binding domain; and a variably spliced mcs domain, which contains within it CS1 and CS5 subdomains. Each domain is composed of repeats denoted as thin rectangles for the type 1 repeats, ovals for the type 2 repeats, and wide rectangles for the type 3 repeats.

In one embodiment, the fibronectin-derived tripeptide sequence Arg-Gly-Asp (RGD) is contemplated. The RGD motif is known for promoting cellular adhesion through binding to integrin receptors, and this interaction has also been shown to play an important role in cell growth, differentiation and overall regulation of cell functions. In one embodiment, RGD peptides with N-terminal cysteine residues are employed as model fibronectin ligands.

When coupled to polyethyleneglycol diacrylate (PEGDA) via a conjugate addition reaction, these peptide-coupled PEGDA solutions containing excess divalent crosslinker were used to crosslink and modify thiolated HA via a second conjugation addition process. Using two kinds of fibroblasts (CF-31 and NIH 3T3) as model cells, the influence of peptide structure (CRGDS and CCRGDS), peptide concentration, PEGDA molecular weight on the cell attachment, spreading, and proliferation was investigated. In addition, a fibroblast-seeded hydrogel formed in vivo by injection of a gelling suspension of fibroblasts resulted in the production of new fibrous tissue in vivo in a nude mouse model.

In certain embodiments, PEG derivatives that are less susceptible to hydrolysis than PEGDA are contemplated, such as PEG-divinylsulfone (PEGDVS) or PEG-diacrylamide.

In one embodiment, a peptide comprising the sequence PHSRN is contemplated. In one embodiment, this PHSRN-containing peptide lacks the α4β1 integrin binding site in the mcs region, but is nonetheless sufficient to stimulate fibroblast invasion of wounds.

### DESCRIPTION OF PREFERRED EMBODIMENTS

1. Peptide Variants In one embodiment, the present invention comprises a peptide derived from fibronectin—but different in sequence. In a preferred embodiment, said peptide comprises the sequence RGD or PHSRN. Of course, the peptide may be larger than three or five amino acids; indeed, the peptide fragment of fibronectin may contain hundreds of additional residues (e.g. five hundred amino acids). One such larger peptide is set forth in U.S. Pat. No. 5,492,890 (hereby incorporated by reference). In one embodiment, the PHSRN-containing peptide is less than one hundred amino acids in length and lacks the RGD sequence characteristic of fibronectin. In another embodiment, the PHSRN-containing peptide is less than one hundred amino acids in length and comprises the RGD sequence. A variety of PHSRN-containing peptides are contemplated, including the PHSRN peptide itself and related peptides where additional amino acids are added to the carboxy terminus, including (but not limited to) peptides comprising the sequence: 1) PHSRN, 2) PHSRNS, 3) PHSRNSI, 4) PHSRNSIT, 5) PHSRNSITL, 6) PHSRNSITLT, 7) PHSRNSITLTN, 8) PHSRNSITLTNL, 9) PHSRNSITLTNLT, 10) PHSRNSITLTNLTP, and 11) PHSRNSITLTNLTPG. Alternatively, PHSRN-containing peptides are contemplated where amino acids are added to the amino terminus, including (but not limited to) peptides comprising the sequence: 1) PEHFSGRPREDRVPHSRN, 2) EHFSGRPREDRVPHSRN, 3) HFSGRPREDRVPHSRN, 4) FSGRPREDRVPHSRN, 5) SGRPREDRVPHSRN, 6) GRPREDRVPHSRN, 7) RPREDRVPHSRN, 8) PREDRVPHSRN, 9) REDRVPHSRN, 10) EDRVPHSRN, 11) DRVPHSRN, 12) RVPHSRN, and 13) VPHSRN. Finally, the present invention contemplates PHSRN-containing peptides where amino acids are added to both the amino and carboxy termini, including (but not limited to) peptides comprising the sequence PEHFSGRPREDRVPHSRNSITLTNLTPG, as well as peptides comprising portions or fragments of the PHSRN-containing sequence PEHFSGRPREDRVPHSRNSITLTNLTPG.

Peptides containing variations on the PHSRN motif are contemplated. For example, the present invention also contemplates PPSRN-containing peptides for use in the above-named assays. Such peptides may vary in length in the manner described above for PHSRN-containing peptides. Alternatively, PPSRN may be used as a peptide of five amino acids.

Similarly, peptides comprising the sequence -HHSRN-, -HPSRN-, -PHTRN-, -HHTRN-, -HPTRN-, -PHSNN-, -HHSNN-, -HPSNN-, -PHTNN-, -HHTNN-, -HPTNN-, -PHSKN-, -HHSKN-, -HPSKN-, -PHTKN-, -HHTKN-, -HPTKN-, -PHSRR-, -HHSRR-, -HPSRR-, -PHTRR-, -HHTRR-, -HPTRR-, -PHSNR-, -HHSNR-, -HPSNR-, -PHTNR-, -HHTNR-, -HPTNR-, -PHSKR-, -HHSKR-, -HPSKR-, -PHTKR-, -HHTKR-, -HPTKR-, -PHSRK-, -HHSRK-, -HPSRK-, -PHTRK-, -HHTRK-, -HPTRK-, -PHSNK-, -HHSNK-, -HPSNK-, -PHTNK-, -HHTNK-, -HPTNK-, -PHSKK-, -HHSKK-, -HPSKK-, -PHTKK-, -HHTKK-, or -HPTKK- are contemplated by the present invention. Such peptides can be used as five amino acid peptides or can be part of a longer peptide (in the manner set forth above for PHSRN-containing peptides).

As noted above, the present invention contemplates peptides that are protease resistant. In one embodiment, such protease-resistant peptides are peptides comprising protecting groups. In a preferred embodiment, the present invention contemplates a peptide containing the sequence PHSRN (or a variation as outlined above) that is protected from exoproteinase degradation by N-terminal acetylation (“Ac”) and C-terminal amidation. The Ac-XPHSRNX-NH₂ peptide (which may or may not have additional amino acids, as represented by X; the number of additional amino acids may vary from between 0 and 100, or more) is useful for in vivo administration because of its resistance to proteolysis.

In another embodiment, the present invention also contemplates peptides protected from endoprotease degradation by the substitution of L-amino acids in said peptides with their corresponding D-isomers. It is not intended that the present invention be limited to particular amino acids and particular D-isomers. This embodiment is feasible for all amino acids, except glycine; that is to say, it is feasible for all amino acids that have two stereoisomeric forms. By convention these mirror-image structures are called the D and L forms of the amino acid. These forms cannot be interconverted without breaking a chemical bond. With rare exceptions, only the L forms of amino acids are found in naturally occurring proteins. In one embodiment, the present invention contemplates PHS(dR)N-containing peptides for wound healing.

2. Mimetics

Compounds mimicking the necessary conformation for recognition and docking to the receptor binding to the peptides of the present invention are contemplated as within the scope of this invention. For example, mimetics of PHSRN peptides are contemplated. A variety of designs for such mimetics are possible. For example, cyclic PHSRN-containing peptides, in which the necessary conformation for binding is stabilized by nonpeptides, are specifically contemplated. U.S. Pat. No. 5,192,746 to Lobl, et al, U.S. Pat. No. 5,169,862 to Burke, Jr., et al, U.S. Pat. No. 5,539,085 to Bischoff, et al, U.S. Pat. No. 5,576,423 to Aversa, et al, U.S. Pat. No. 5,051,448 to Shashoua, and U.S. Pat. No. 5,559,103 to Gaeta, et al, all hereby incorporated by reference, describe multiple methods for creating such compounds.

Synthesis of nonpeptide compounds that mimic peptide sequences is also known in the art. Eldred, et al, J. Med. Chem. 37:3882 (1994) describe nonpeptide antagonists that mimic the Arg-Gly-Asp sequence. Likewise, Ku, et al, J. Med. Chem. 38:9 (1995) give further elucidation of the synthesis of a series of such compounds. Such nonpeptide compounds that mimic PHSRN peptides are specifically contemplated by the present invention.

The present invention also contemplates synthetic mimicking compounds that are multimeric compounds that repeat the relevant peptide sequence. In one embodiment of the present invention, it is contemplated that the relevant peptide sequence is Pro-His-Ser-Arg-Asn or Pro-Pro-Ser-Arg-Asn. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to the above-described PHSRN-containing peptides (including, but not limited to, peptides in which L-amino acids are replaced by their D-isomers). One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant. W. R. Pearson and D. J. Lipman, Proc. Natl. Acad. Sci. (USA), 85:2444-2448 (1988); D. J. Lipman and W. R. Pearson, Science, 227:1435-1441 (1985). In the present invention, synthetic polypeptides useful in wound healing are those peptides with statistically significant sequence homology and similarity (Z value of Lipman and Pearson algorithm in Monte Carlo analysis exceeding 6).

3. Formulations

It is not intended that the present invention be limited by the particular nature of the therapeutic preparation, so long as the preparation comprises a functional fragment or domain of fibronectin attached through a linker to HA. For example, such compositions can be provided together with physiologically tolerable liquid, gel or solid carriers, diluents, adjuvants and excipients.

These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

Such compositions are typically prepared as liquid solutions or suspensions, or in solid forms. Formulations for wound healing usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compositions are also prepared as injectables, either as liquid solutions or suspensions; solid forms suitable for solution in, or suspension in, liquid prior to injection may also be prepared.

The compositions of the present invention are often mixed with diluents or excipients which are physiological tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

4. Dermal Fillers, Substitutes and Implants

While the compositions of the present invention have been discussed extensively in the context of wound healing, the present invention also contemplates that the compositions can be employed in dermatology to treat, wrinkles, folds, scars, and to enhance tissue (e.g. lip enhancements). The compositions (e.g. through one or more simple injections beneath the surface of the skin) help to “fill out” and smooth away lines, wrinkles, scars or other folds (including deep facial folds, frown lines, peri-oral lines and naso-labial folds). In one embodiment, the present compositions are used following cosmetic surgery (e.g. face lifts) to enhance surgical results.

In one embodiment, the compositions of the present invention (described above in the context of either attached peptides or attached domains), are placed in or under the skin. In another embodiment, such compositions are first “seeded” with suitable cells (e.g. fibroblasts, keratinocytes, etc.). In one embodiment, the cells used to seed the compositions of the present invention are from the subject who is receiving the dermal fill, dermal substitute, soft tissue augmentation, or dermal implant (thereby avoiding immune reactions). In some embodiments, anchoring structures known in the art are employed in the context of using the compositions of the present invention. In one embodiment, it is preferred that the injected and implanted compositions are free of exogenous collagen.

The compositions of the present invention have been described above in the context of either attached peptides or attached domains. However, in the context of dermal fills, substitutes and implants, the present invention also contemplates embodiments lacking the attached peptides or attached domains. In one embodiment, the composition simply comprises a PEG-GAG (glycoaminoglycan) conjugate (e.g. PEG-HA) for such dermatological and cosmetic applications. In one embodiment, fibronectin-free compositions resulting from the combinations of either of two kinds of thiolated hyaluronan (HA-DTPH and HA-DTBH) with any one of four kinds of α, β unsaturated esters and amides of PEG (PEG-diacrylate (PEGDA), PEG-dimethacrylate, PEG-diacrylamide, and PEG-divinylsulfone) are contemplated as useful for dermal fill, skin substitutes, and tissue implants. The present invention contemplates methods wherein such compositions are place on the skin or injected underneath the skin. The present invention also contemplates methods of synthesis and/or manufacture. In one embodiment, the method comprises a) modifying carboxylic groups of HA to contain free thiol groups so as to create thiolated HA; b) reacting said thiolated HA with a derivative of PEG (e.g. an α, β unsaturated derivative of PEG such as PEGDA) to create a conjugate (e.g. a crosslinked conjugate). In one embodiment, step b) is done through Michael addition.

Experimental

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); μmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar);° C. (degrees Centigrade); mAb (monoclonal antibody); MW (molecular weight); PBS (phophate buffered saline); U (units); d(days).

The materials and methods used in the Examples include the following:

Materials

Fermentation-derived hyaluronan (HA, sodium salt, M_{w} 1.5 MDa) was provided by Clear Solutions Biotechnology, Inc. (Stony Brook, N.Y.). 1-Ethyl-3-[3-(dimethylamino)propyl]-carbodiimide (EDCI), acryloyl-chloride, poly(ethylene glycol) (Mw 3400 and 1000 Da), and poly(ethylene glycol) diacrylate (Mw 700 Da) (PEGDA 700), were purchased from Aldrich Chemical Co. (Milwaukee, Wis.). Poly(ethylene glycol) divinyl sulfone (Mw 3400 Da) was purchased from Nektar Therapeutics, Huntsville, Ala. Dulbecco's phosphate buffered saline (DPBS) was obtained from Sigma Chemical Co. (St. Louis, Mo.). Dithiothreitol (DTT) was purchased from Diagnostic Chemical Limited (Oxford, Conn.). 5,5′-dithio-bis(2-nitrobenzoic acid) (DTNB) was purchased from Acros (Houston, Tex.). The 3,3-dithiobis(propanoic dihydrazide) (DTPH) modified HA derivative (HA-DTPH) was synthesized as described by Shu, et al., Biomacromolecules 3:1304, 2002. The pKₐ of thiols was determined to be 8.87; the free thiol content was 42 thiols per 100 disaccharide units; and the molecular weight was determined by calibrated gel permeation chromatography to be Mw 158 KDa, Mn 78 KDa, and polydispersity index 2.03. Poly(ethylene glycol) diacrylate (PEGDA 3400 and 1000) was synthesized from Poly(ethylene glycol) (Mw 3400 and 1000 Da respectively) according to Nakayama and Matsuda, J. Biomed. Mater. Res. 48:511, 1999. Degree of substitution for PEGDA 3400 was 95% and for PEGDA 1000 was 93%. Two cell adhesion peptides with Arg-Gly-Asp (RGD) sequence and cysteine residue(s) (CRGDS, CCRGDS) and one scrambled peptide (CRDGS) were kindly supplied by Dr. Alyssa Panitch (Department of Bioengineering, Arizona State University).

Analytical Instrumentation

Proton NMR spectral data were obtained using a Varian INOVA 400 at 400 Hz. UV-vis spectral data were obtained using a Hewlett Packard 8453 UV-visible spectrophotometer (Palo Alto, Calif.). Gel permeation chromatography (GPC) analysis was performed using the following system: Waters 515 HPLC pump, Waters 410 differential refractometer, Waters™ 486 tunable absorbance detector, Ultrahydrogel 250 or 1000 columns (7.8 mm i.d.×130 cm) (Milford, Mass.). Eluent was 200 mM phosphate buffer (pH 6.5)/MeOH=80:20 (v/v) and the flow rate was 0.3 or 0.5 m/min. The system was calibrated with standard HA samples provided by Dr. U. Wik (Pharmacia, Uppsala, Sweden). Fluorescence images of viable cells were recorded using a confocal microscopy (LSM 510, Carl Zeiss Microimaging, Inc. Thornwood, N.Y.). Cell proliferation was determined by MTS (Cell-Titer 96 Proliferation Kit, Promega, Madison, Wis.) or MTT (Sigma) assay at 550 nm, which was recorded on an OPTI max microplate reader (Molecular Devices, Sunnyvale, Calif.).

Analysis of the Conjugate Addition Reaction

The reaction kinetics of conjugate addition of RGD peptides with cysteine residue to PEGDA was followed using the reagents DTNB (Arpicco, et al., Bioconjug. Chem. 8:327, 1997) or NTSB (Gopalakrishna, et al., Arch. Biochem. Biophys. 348:25, 1997). CRGDS (3.6 mg, 0.0067 mmol), CCRGDS (4.3 mg, 0.0067 mmol) or CRDGS (3.6 mg, 0.0067 mmol) and PEGDA (PEGDA 3400 223.5 mg, 0.067 mmol; PEGDA 1000 72 mg, 0.067 mmol; or PEGDA 700 42.5 mg 0.067 mmol) were dissolved in 5 ml 0.1 N phosphate buffer saline (PBS) pH 7.4, and then the consumption of thiols was monitored by DTNB or NTSB.

Hydrogel Preparation

PEGDA solutions. PEGDA (PEGDA 3400, 223.5 mg, 0.067 mmol; PEGDA 1000, 72 mg, 0.067 mmol; or PEGDA 700, 42.5 mg 0.067 mmol) without or with peptides, CRGDS (3.6 mg, 0.0067 mmol), CCRGDS (4.3 mg, 0.0067 mmol) or CRDGS (3.6 mg, 0.0067 mmol), were dissolved in 5 ml DPBS solution, and stirred for 4 h. In the above stock solutions the molar ratio of peptides to PEGDA was 1/10. PEGDA solutions with lower peptide concentration (peptides/PEGDA: 5/100, 1/100 and 1/500) were prepared by diluting the stock solution with blank PEGDA solution. Then the solutions were sterilized by filtering through 0.45 μm filter.

HA-DTPH solutions. HA-DTPH was dissolved in serum free DMEM/F-12 medium or complete DMEM/F-12 medium supplied with 10% new-born calf serum (for NIH 3T3 fibroblast) or fetal bovine serum (for CF-31 fibroblast), 2 mM L-glutamine and 100 units/ml antibotic-antimycotic (GIBCO BRL, Life Technologies, Grand Island, N.Y.), and 50 μg/ml ascorbic acid (Sigma) to give a 1.25% (w/v) solution, and the solution pH was adjusted to 7.4 by adding 1.0 M NaOH. Then the solutions were sterilized by filtering through a 0.45 μm filter.

PEGDA-linked Hydrogel preparation. In a laminar flow hood, 1 ml of PEGDA solution with different concentrations of synthetic peptides or proteins (functional domains of fibronectin) was added into 4 ml of HA-DTPH solution (the ratio of thiols to acrylate was ca. 2:1), and mixed for 30 seconds. Then, 0.3 ml mixture solution was injected into each well of 24-well plates. Usually the solution gelled within ca. 7 min. After 1 h, the hydrogels were used directly in the following experiments or dried in the hood for 2 days to give films.

PEGDVS-linked hydrogel preparation. The final concentration of HA-DTPH was 1% (w/v) and the concentration of PEGDVS was 4.5% (w/v), creating a ratio that leaves >90% of the PEGDVS cross-linked at each end (Shu, et al., Biomaterials, 2004) when equilibrium is reached. Different concentrations of proteins were chosen as experimental conditions required.

Protein Adsorption Studies

Adsorption of serum proteins to HA-DTPH/PEGDA hydrogels was evaluated by modifications of methods described extensively by Nuttelman et al. (Biomed. Sci. Instrum. 35:309, 1999; Biomaterials 23:3617, 2002; J. Biomed. Mater. Res. 57:217, 2001; ibid, 68:773, 2004). HA-DTPH/PEGDA hydrogels prepared in serum free DMEM/F-12 medium were rinsed 4 times with serum free DMEM/F12 medium over 1 h. Next, 0.5 ml new-born calf serum (GIBCO) was added on the top of hydrogel in 24-well plates and incubated at 37° C. for 45 min. Then, the hydrogels were rinsed with DPBS thoroughly to remove the unabsorbed proteins. The hydrogels were incubated with 1 ml graded isopropanol solutions (10, 30, 50 and 70% in distilled water) at room temperature for 20 min to remove the absorbed proteins. The washes were decanted and transferred to 1 ml conical tubes and solvents removed by evaporation overnight and then lyophilized for SDS-PAGE analysis.

In Vitro Cell Spreading and Proliferation

Cell culture. Dermal fibroblasts from a 31-yr old Caucasian female (CF-31) (Clonetics, San Diego) and NIH 3T3 fibroblast (ATCC) were routinely cultured in completed DMEM/F-12 medium supplied with 10% new-born calf serum (for NIH 3T3 fibroblast) or fetal bovine serum (for CF-31 fibroblast), 2 mM L-glutamine and 100 units/ml antibotic-antimycotic (GIBCO BRL, Life Technologies, Grand Island, N.Y.), and 50 μg/ml ascorbic acid (Sigma) at 37° C. in a humidified 5% CO₂ incubator. Monolayers of fibroblasts in their growth phase (ca. 90% confluence) were dissociated with trypsin/1 mM EDTA in DPBS, centrifuged and resuspended in the serum free medium or complete medium.

Cell spreading on the RGD-peptides coupled HA-DTPH/PEGDA hydrogel surface. HA-DTPH/PEGDA hydrogels or hydrogel films in 24-well plate were rinsed with serum free medium or complete medium 3 times, and then CF-31 and NIH 3T3 fibroblasts were seeded on the surface of the hydrogel or hydrogel film surface at a density of 4×10⁴/well. Then, 1 ml of serum free medium or complete medium was added into each well, and the cells were cultured at 37° C. in a humidified 5% CO₂ incubator. At 2, 4, 8 and 18 h post seeding, 0.4 ml of 10% formalin solution (Sigma) was added into each well to fix cell for 15 min. The cells were then stained at room temperature with 0.1% crystal violet in 200 mM boric acid (pH 8.0) for 15 minutes. Photomicrographs of the cytoplasmic-stained cells were taken a 100×total magnification using an inverted Nikon microscope with a CCD camera on a minimum of five random fields per well (n=3 per composition). The bundled Spot 3.0 software (Diagnostic Instruments, Sterling Heights, MI) was used to classify the cell morphology into 3 categories viz. round, partially spread or fully spread as reported by Neff etc[47]. It was defined that cells with surface area <2000 pixels² were completely round, 2000 pixels² <cells with area <8000 pixels² had few cytoplasmic extensions although they maintained a somewhat round appearance, and cells with surface areas >8000 pixels² were fully spreading and had multiple cytoplasmic extensions in various directions.

Cell spreading assay to determine hydrogel degradation by hydrolysis. PEGDA-crosslinked and PEGDVS-crosslinked hydrogels were prepared as described, and each was conjugated with cellular FNIII₍₈₋₁₁₎(the RGD-mediated cell binding domain) at low titer (0.05 μM) to maximize assay sensitivity. Hydrogel samples were incubated in the wells of 24-well plates in a humidified 5% CO₂ environment at 37° C. in PBS for 0, 6, 9, or 12 days (with daily changes of PBS). At the end of each sample's incubation period, the sample was seeded with CF-31 cells at a density of 17,500 cells/cm². Six hours later, the cells in each instance were formalin-fixed, stained with crystal violet and photographed through a 40× microscope objective. The morphology of the cells (“round,” “partially spread,” or “fully spread”) was analyzed with the aid of Bundled Spot™ software (V. 3.0), which measures, in pixels, the area occupied by a test cell and utilizes the measurement as a basis for assigning the cell to one of three categories (fully spread, fully rounded, or partially rounded).

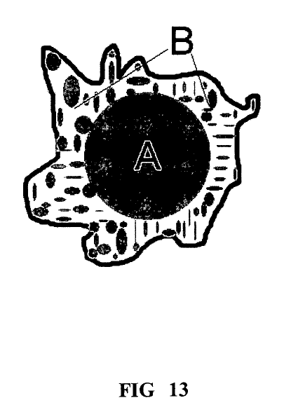

Out-migration assay of hydrogel/fibroblast compatibility. The agarose droplet migration assay, essentially as described by Varani, et al., (Amer. J. Pathol. 90: 159-178, 1978) and schematically depicted in FIG. 13, was adapted to provide an index of compatibility between hydrogel surfaces and fibroblasts migrating thereon. PEGDA-linked and PEGDVS-linked hydrogels were prepared as described above. Each was conjugated with all three of the functional domains of FN at high titer (0.26 μM), so that differences intrinsic to the hydrogel itself would tend to dictate differences in outcome (low titers would be preferred where the migratory potential intrinsic to different fibroblasts is to be evaluated). Hydrogels were cured (allowed to reach cross-link equilibrium) overnight at 4° C. For each experiment, 1.1×10⁴ fibroblasts were dispersed in 1 μl of a 0.2% solution of agarose which was dotted as a droplet onto the hydrogel surface. The agarose was allowed to set (incubation at 4° C. for 20 minutes) and was then covered with serum-free DMEM and PDGF (final concentration=30 ng/ml). Eighteen hours later, each preparation was fixed, stained with crystal violet, photographed through a stereo microscope and analyzed with the Bundled Spot software by subtracting from the total pixels occupied by cells the pixels occupied by the initial agarose droplet.

Cell proliferation on the RGD-peptide coupled HA-DTPH/PEGDA hydrogel surface. Peptide-coupled HA-DTPH/PEGDA hydrogels or hydrogel films in 24-well plates were rinsed 3 times with complete medium, and then CF-31 and NIH 3T3 fibroblasts were seeded on the surface of the hydrogel or hydrogel film surface at a density of 1×10⁴/well. After that 1 ml of complete medium was added into each well, and the cells were cultured at 37° C. in a humidified 5% CO₂ incubator and the medium was changed daily. At different time points (day 1, 2, 3 and 4), the cell number was determined by MTS assay (Cell-Titer 96 Proliferation Kit, Promega, Madison, Wis.) as previously described Shu, et al., Biomacromolecules 3:1304, 2002; Ren and Tang, Anticancer Res. 19:403, 1999) and the absorption recorded at 550 nm with an OPTI Max microplate reader (Molecular Devices). Cell numbers were obtained using standard curves generated from the assay.

Cell proliferation inside the RGD-peptide coupled HA-DTPH/PEGDA hydrogel. NIH 3T3 fibroblasts were suspended in 1.25% (w/v) HA-DTPH solution in complete medium (pH 7.4) at a density of 1×10⁶/ml, and then 1 ml of stock peptide-coupled PEGDA or blank PEGDA solution (see hydrogel preparation) was added into 4 ml of cell/HA-DTPH mixture solution. After mixed, 200 μl of the mixture of HA-DTPH/PEG-diacrylate/T31 fibroblasts was injected into each well of 24-well plates. After 1 h, 1.0 ml of complete medium was added into each well and incubated in 37° C. 5% CO₂ incubator. The medium was changed every day without damaging the hydrogel. The viability of NIH 3T3 fibroblast inside hydrogel following 1, 5 and 15 d in vitro culture was determined by a double-staining procedure (Saha, et al., Dev. Comp. Immunol. 27:351, 2003) using fluorescein diacetate (F-DA) and propidium iodide (PI). Briefly, cell-hydrogel constructs were rinsed twice with DPBS buffer, stained with F-DA (0.02 mg/ml)(Molecular Probes, Eugene, Oreg.) and PI (0.2 μg/ml)(Sigma) at room temperature for 3 min, rinsed twice with DPBS buffer, stored on ice, and observed under confocal microscope (Zeiss Axioplan 2 Imaging System, LSM 5 Pa).

The cell proliferation inside hydrogel following in vitro culture of day 1, 5, 10 and 15 was determined by MTT (3-[4,5-dimethylthiazol-2-yl]-2,5-diphenyltetrazolium bromide) assay (Sigma). A 1% MTT in serum-free media solution was added to each well containing the cell-hydrogel construct. Active mitochondria metabolize the tetrazolium salt to form an insoluble formazin dye. After 8 h of incubation, the constructs were placed in 10 ml glass vials and 2 ml 0.04 N HCl in isopropanol was added to extract the formazin. The absorbance was recorded at 550 nm with an OPTI Max microplate reader (Molecular Devices) and was converted into a cell number based on standard curves.

In vivo Studies

RGD-peptide coupled HA-DTPH/PEGDA hydrogel for injectable fibrous tissue regeneration in nude mouse. Animal experiments were carried out according to NIH guidelines for the care and use of laboratory animals (NIH publication #85-23 rev. 1985). Male nude mice (n=12) (Simonsen Laboratories Inc., Gilroy, Calif.), 4-6 weeks old were anesthetized with 2.5% isoflurane using a VetEquip inhalation anesthesia system (Pleasanton, Calif.). NIH 3T3 fibroblasts were suspended in 1.25% (w/v) HA-DTPH solution in complete medium (pH 7.4) at a density of 50×10⁶ cell/ml, and then 1 ml stock CRGDS-coupled PEGDA (peptide concentration 10/100) or blank PEGDA (see 2.4 hydrogel preparation) was added into 4 ml cell/HA-DTPH mixture solution and mixed. Each nude mouse received four 300 μl s.c. dorsal injections by means of an 18-gauge needle under the dorsal panniculus camosus (two 300 μl solution with cells and two 300 μl solution without cells as control). At each time point (4 and 8 weeks after implantation), two nude mice were sacrificed, and the implants with the surrounding tissues were removed from the mouse, fixed in 10% buffered formalin solution (Sigma) for 24 h, embedded in paraffin, cut into 5-μm sections, and mounted onto slides.

Histological and immunohistochemical staining. The slides were deparaffinized and rehydrated, and then washed with Tris-buffered saline (TBS), and then incubated in 3% hydrogen peroxide for 5 min. After being rinsed with TBS, samples were treated with Proteinase-K enzyme for 5 min, rinsed again with TBS for 5 min and followed by 15 min incubation with anti-procollagen (1:100, Chemicon International, Inc., Temecula, Calif.). The samples were then treated with the DAKO-LSAB kit (Dako): biotin 10 min; TBS rinse and StreptAvidin 10 min. After that the slides were again rinsed with TBS and treated with a 5 min DAB substrate solution (Research Diagnostics), rinsed with water and covered slipped with hematoxylin (Dako).

Statistical Analysis

Statistical analysis was performed using a two-tailed, unpaired Student's t-test. P-values less than 0.05 were considered to be significant.

### EXAMPLE 1

It was suggested in U.S. Pat. No. 6,194,378 that certain fragments of fibronectin should be directly attached to hyaluronic acid using methodology whereby hyaluronic acid is derivatized using dihydrazide (according to the teachings of U.S. Pat. Nos. 5,652,347 and 5,616,568). This idea was tested using a migration assay in which fibroblasts migrate from collagen-coated beads (Cytodex-3 beads) into various crosslinked HA constructs.

The method for making a functionalized hyaluronate involves providing hyaluronate in an aqueous solution, mixing the hyaluronate in aqueous solution with a dihydrazide to form a hyaluronate-dihydrazide mixture, adding a carbodiimide to the hyaluronate-dihydrazide mixture and allowing the hyaluronate and dihydrazide to react with each other in the presence of the carbodiimide under conditions producing hyaluronate functionalized with dihydrazide. The hyaluronate functionalized with dihydrazide has a pendant hydrazido group which is useful in subsequent reactions. It was discovered that when dihydrazides are first added to the hyaluronate followed by addition of carbodiimide, the dihydrazide adds to the O-acylurea before it undergoes rearrangement to the more stable N-acylurea.

The functionalizations of HA with dihydrazides are preferably carried out under mild conditions including a pH of about 2 to 8 preferably about 3 to 6. The hyaluronate is dissolved in water which may also contain water-miscible solvents such as dimethylformamide, dimethylsulfoxide, and hydrocarbyl alcohols, diols, or glycerols. At least one molar equivalent of dihydrazide per molar equivalent of HA is added. For maximum percentage functionalization, a large molar excess of the dihydrazide (e.g., 10-100 fold) dissolved in water or aqueous-organic mixture is added and the pH of the reaction mixture is adjusted by the addition of dilute acid, e.g., HCl. A sufficient molar excess (e.g., 2 to 100 fold) of carbodiimide reagent dissolved in water, in any aqueous-organic mixture, or finely-divided in solid form is then added to the reaction mixture. It is important that the hyaluronate and dihydrazide be mixed together before addition of the carbodiimide. An increase in pH may be observed after addition of the carbodiimide and additional dilute HCl or other acid may be added to adjust the pH. The reaction is allowed to proceed at a temperature of about 0 degrees C. to about 100 degrees C. (e.g., just above freezing, to just below boiling), preferably at or near ambient temperatures for purposes of convenience. The time of the reaction is from about 0.5 to about 48 hours, preferably about one to about five hours with periodic testing and adjusting of the pH until no further change in pH is observed. The pH may then be adjusted to an approximate neutral range and the product, which is hydrazido functionalized hyaluronate, may be concentrated and purified by methods known in the art such as dialysis, rotary evaporation at low pressure and/or lyophilization. There are three commercially available dihydrazides: succinic, adipic, and suberic. Each can be attached to the HA backbone.

Following the attachment to the derivatized HA, the functionality of the construct was tested. In the first experiment, the cells would not outmigrate and simply rounded up on the HA construct where fibronectin domains were directly attached to dihydrazide derivatized HA (“HAFN”). To avoid any question of toxicity by residual components generated in the covalent attachment chemistry, the constructs were redialyzed. The HA gel migration assay was repeated to determine whether the cells would now outmigrate onto the redialyzed HAFN construct. The cells did not move out onto the redialyzed HAFN, but once again rounded up on the Cytodex-3 beads.

The results might be explained in a number of ways. First, it is possible the HAFN construct contained non-soluble chemicals that cause contact cytotoxicity. Second, the crosslinking conditions may have been such that the fibronectin became overly crosslinked. Too strong of a crosslink may provide a danger that the critical domains of fibronectin will become masked or distorted. Third, fibronectin might have been denatured by the crosslinking reaction or cell ligand sites blocked, thus preventing cell interactions with the matrix.

### EXAMPLE 2

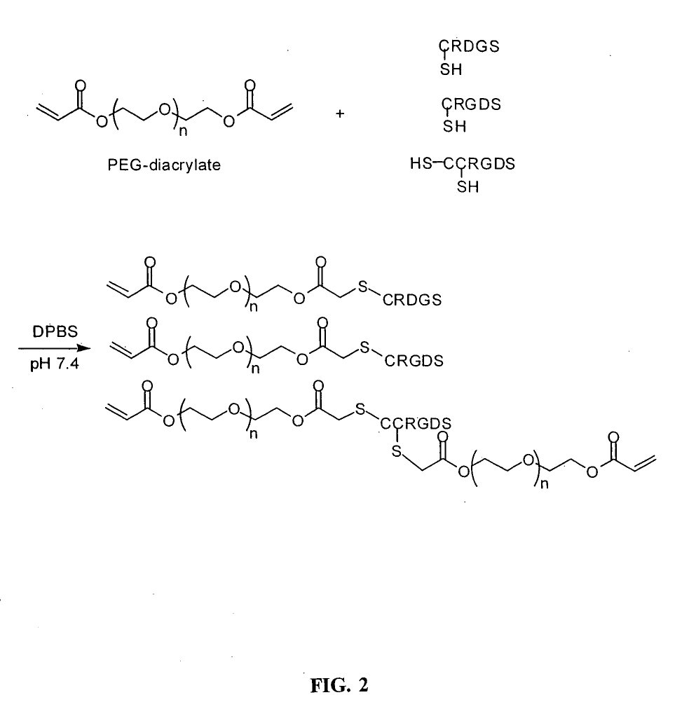

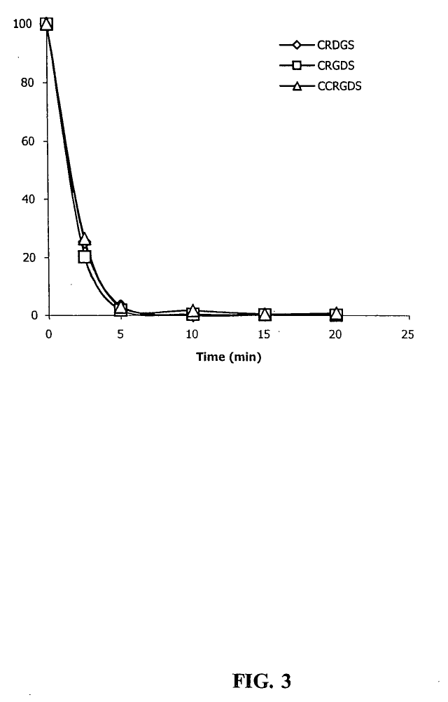

In view of the results described in Example 1, peptides containing native human fibronectin sequences were covalently attached in an indirect manner to HA. In this example, the RGD sequence of fibronectin was first conjugated to PEGDA. More specifically, RGD peptides with either one or two cysteine residue (CRGDS and CCRGDS) were conjugated to an excess of PEGDA to give a mixture of unreacted homobifunctional PEGDA plus monofunction peptide-PEG acrylate. A nonsense peptide (CRDGS) was used as the control (FIG. 2). During the first conjugation (i.e. during the addition of peptide to PEGDA), the molar ratio of thiols to acrylates was controlled at 1:20 (CRGDS and CRDGS) or 1:10 (CCRGDS), and the conjugation was complete within 5 minutes (FIG. 3). Under these conditions, only a portion of the acrylate groups were targeted for reaction with peptides, and sufficient amounts of unmodified PEGDA remained to crosslink HA-DTPH. Using this protocol, it was unnecessary to isolate the monovalent CRGDS-PEG acrylate, CRDGS-PEG acyrlate, or divalent CCRGDS bis(PEG acrylate) present in the crosslinking mixture.

Under sterile conditions, HA-DTPH/PEGDA hydrogels with different concentration of RGD peptides were fabricated by adding 1 ml of peptide-coupled PEGDA solution into 4 ml of HA-DTPH solution (1.25% w/v), maintaining a two-fold molar ratio of thiol to acrylate functionalities. This ratio ensures complete reaction of the PEGDA and the peptide-PEG acrylate derivative. GPC analysis indicated that little free PEGDA remained (results not shown), and thus the RGD peptide PEG-acrylate was successfully coupled into the hydrogel.

HA-DTPH/PEGDA hydrogel prepared in serum free DMEM/F-12 medium was treated with new-born calf serum (GIBCO) and incubated at 37° C. for 45 min. Then, the unadsorbed proteins were removed by thoroughly rinsing with DPBS, and the absorbed proteins were collected by graded isopropanol solution rinsing (10, 30, 50 and 70% in distilled water). The rinsing solution was then lyophilized for SDS-PAGE analysis and the results indicated that only very low amounts of protein were adsorbed to the hydrogel (results not shown).

Since the HA-DTPH/PEGDA hydrogel was non-adhesive to proteins, when fibroblasts (CF-31) were seeded onto the hydrogel surface in serum free medium, the cells failed to attach and retained a rounded shape, later aggregating into large clusters. The coupling of RGD peptides promoted cell attachment and spreading even in serum free medium and this was found to be peptide concentration dependent (data not shown). On the other hand, the scrambled peptide (CRDGS) failed to promote cell spreading, and all cells were rounded at all concentrations in serum free medium (pictures not shown). These results indicated that the cell spreading was specifically in response to the RGD sequence.

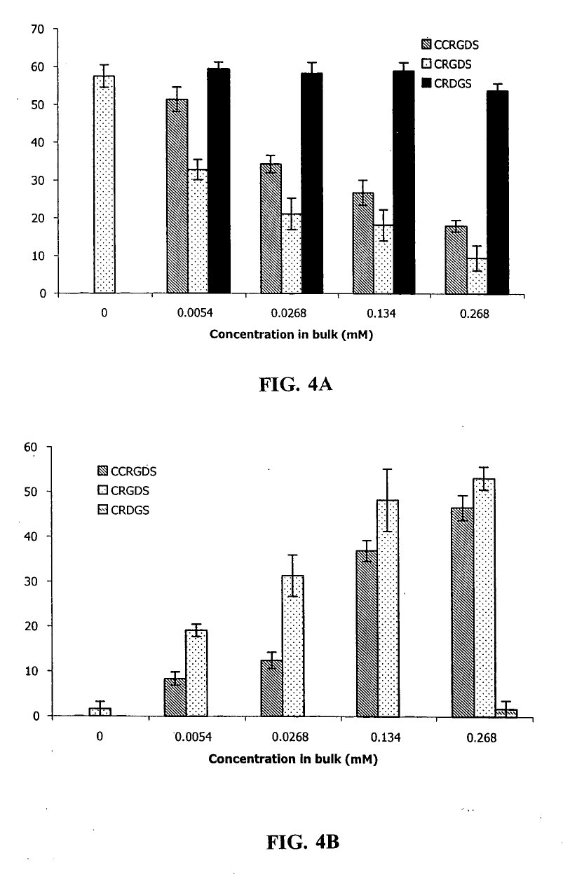

FIG. 4 shows the influence of RGD peptide concentration and structure on the CF-31 fibroblasts spreading on the hydrogel surface in complete medium at 18 h post seeding. Similar to the results in serum free medium, without RGD peptides most of cells (ca. 58%) remained rounded (FIG. 4a) and less than 2% percent cells spread (FIG. 4 b). However, with the increase of CRGDS concentration in bulk from 0.0054 to 0.268 mM, the percentage of spreading cells increased from 19.1 to 53.0% (FIG. 4 b) and accordingly the percentage of round cells decreased from 51.2 to 18% (FIG. 4 a).

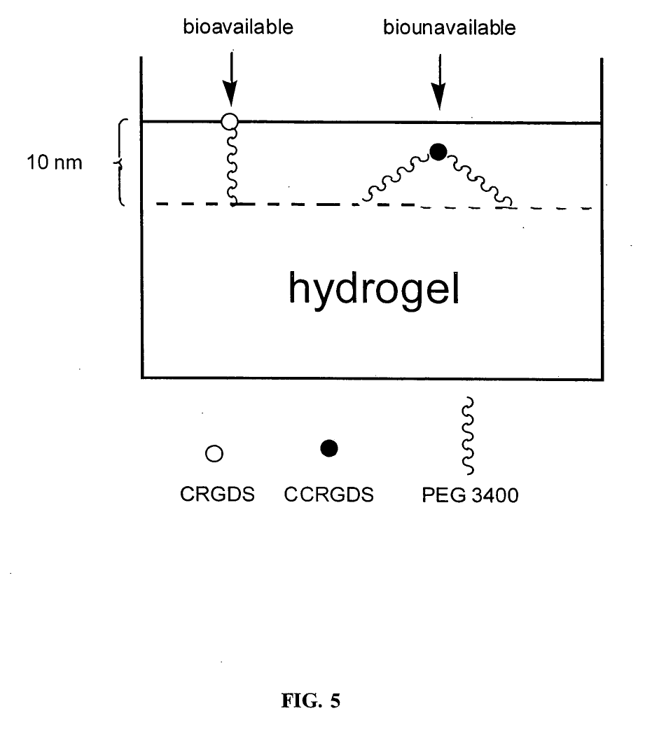

From FIG. 4, it also can be seen that under the same condition the cell spreading percent on CCRGDS-modified hydrogel surface was lower than on CRGDS-modified hydrogel surface. For instance, with the CRGDS concentration in bulk 0.0054, 0.0268, 0.134 and 0.268 mM, the percentage of spreading cells for CRGDS is 19.1, 31.3, 48.2 and 53.0%, while the value was 8.3, 12.4, 36.8 and 46.5% for CCRGDS, which is statistically significant (p<0.05). This result suggests that the structure of RGD peptides with influences biological function. Usually the biological ligand density on surface is the major factor that controls cell spreading. Although in both cases, the bulk RGD density was the same, the surface RGD density perceived by the cells appears different for CCRGDS and CRGDS. As reported by Hem and Hubbell, the peptide surface concentration could be estimated from bulk concentration based on the assumption that the peptide in the outer 10 nm (based on the molecular length of the spacer) of the hydrogel was bioavailable when PEG 3400 was used as a spacer. However, in our experiment, CCRGDS would have two PEG 3400 spacers and two reactive linker sites attached to the peptide. As a bifunctional adduct, the bis(acrylate-PEG)CCRGDS adduct effectively acted as a crosslinker and not as a surface-pendant free RGD ligand. The two spacers of CCRGDS also may retard the peptide flexibility and reduce the integration efficiency of peptide to integrin receptor of the cells. Thus, the surface RGD density of CCRGDS was lower than that of CRGDS (FIG. 5) and the peptide is also less available for cell recognition.

Under the same conditions, NIH 3T3 fibroblasts were more sensitive to the RGD peptides on the hydrogel surface than CF-31 fibroblast, and the spreading percentage for NIH 3T3 was higher than that of CF-31. For example, with the RGD concentration in bulk 0.268 mM more than 90% cells spread at 4 h post-seeding and the cells even more spreading at 8 h, while for the blank or the scramble peptide (CRDGS) coupled hydrogel the cells were rounded (data not shown). The cell area in the case of CRGDS at 4 and 8 h was larger than that for CCRGDS due to the higher surface peptide density.

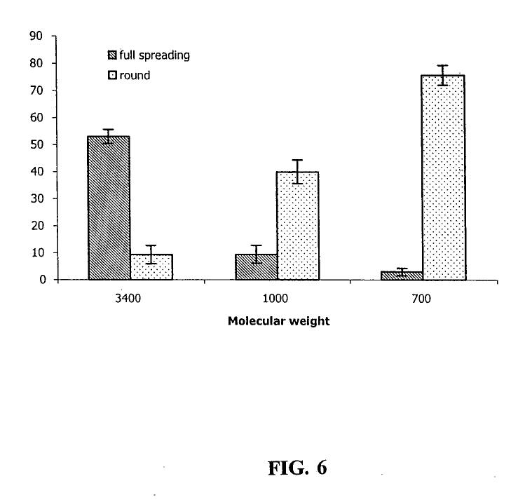

Under the same conditions, the difference in the structure of CRGDS and CCRGDS resulted in the different surface peptide density and thus cell spreading (FIGS. 4 and 5). On the other hand, the molecular weight of PEG spacer also substantially influenced the surface peptide density. Under the same conditions (peptide concentration in bulk 0.268 mM) CRGDS-coupled PEGDA with molecular weight 700 and 1000 were also used to fabricate hydrogel, and CF-31 fibroblasts were seeded on the hydrogel surfaces (PEG 700, 1000 and 3400) and the cell spreading was evaluated. According to the assumption that that the peptide in the outer 10 nm of the hydrogel was bioavailable with PEG 3400 spacer, with the PEG 700 and 1000 linkers only the peptides in the outer 2.1 nm and 2.3 nm would be calculated to be bioavailable, and the calculated RGD surface density of the unswollen hydrogel with PEG 700, 1000 and 3400 was calculated to be 0.056×10⁻³, 0.062×10⁻³, 0.268×10⁻³ pmol/cm². Accordingly, the spreading percent of CF-31 fibroblast increased significantly from ca.3.0 to 53% while the round cell percent decreased from 75.6 to 9.4% as the PEG spacer molecular weight increased from 700 to 3400 (FIG. 6).

Compared to other PEGDA-based hydrogels, the sensitivity of the cell response to the RGD peptide in these HA-DTPH/PEGDA hydrogels is considerably higher, and cell spreading occurred although the surface peptide density in our hydrogel was several orders of magnitude lower that those normally employed. For example, in photopolymerized PEGDA hydrogels, there are many single-end unreacted oligomers (often >10%) remained and that will mask the surface RGD peptide and block the access of the peptide to cell integrin-binding receptor, while in our hydrogel the previous results showed that the single-end unreacted PEGDA is much lower (<7%), and the masking effect is less apparent.

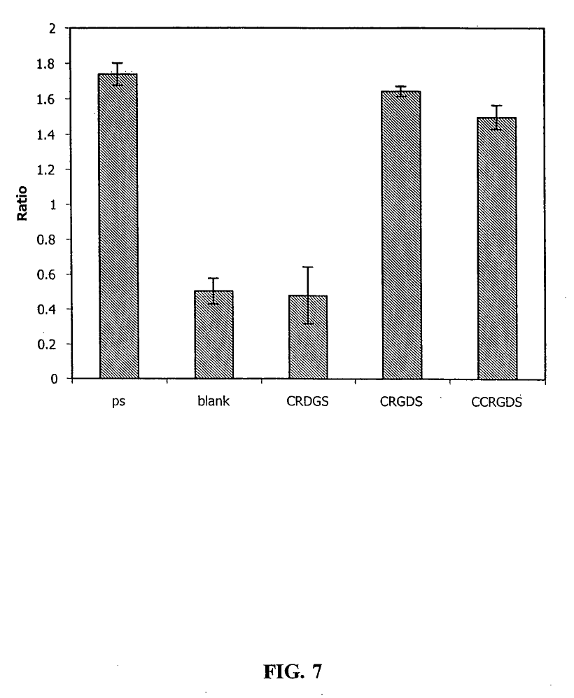

The cell proliferation was further investigated by seeding fibroblasts on the hydrogel surface (PEGDA 3400) and culturing in vitro for up to 4 days. At different time points, the medium was aspirated and the cell number was determined by MTS assay. In 24 h, with the peptide concentration in bulk 0.268 mM, NIH 3T3 fibroblasts on CRGDS-coupled hydrogel surface proliferated 1.64-fold, slightly higher than that on CCRGDS-coupled hydrogel surface (1.50-fold) (P<0.05), which was comparable with cell culture polystyrene surface (1.74-fold). On the blank and nonsense peptide (CRDGS) hydrogel surfaces, no spreading or proliferation was observed (FIG. 7).

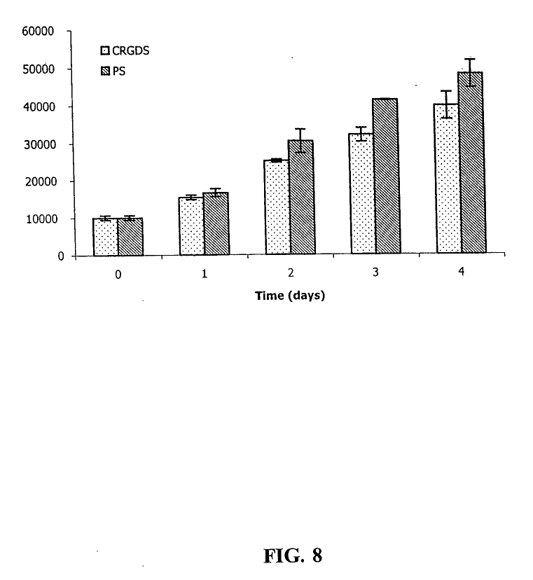

FIG. 8 shows the proliferation of NIH 3T3 fibroblast proliferation following culture in vitro for 4 days on the CRGDS coupled hydrogel surface using PEGDA 3400 (CRGDS concentration in bulk 0.268 mM), with cell culture polystyrene as the control. The proliferation on the hydrogel film surface was comparable to the control, and the cell density was ca. 80% of the control. Microscopy observation revealed that the fibroblasts eventually covered the surface of the hydrogel film surface after 4 days of culture.

Finally, NIH 3T3 fibroblasts were encapsulated in situ inside the hydrogels at a density 1×10⁶/ml, and cultured in vitro for 15 days. At different time points, the cell number inside the hydrogel was evaluated by MTT assay. A transient decrease of cell density (usually ca.30%) occurred after 1 day in vitro culture, with the appearance of some dead cells according to the FDA/PI double staining protocol. Nonetheless, the majority of cells survived the in situ crosslinking and encapsulation procedure. After in vitro culture for 5, 10 and 15 days, very few dead cells were observed and the cell density increased steadily. For instance, in the blank hydrogel the cell density increased 2-fold at day 15 compared to day 1. Interestingly, RGD peptides inside the hydrogel only modestly increased cell proliferation, and the cell proliferated 253% for CCRGDS and CRGDS, while for the nonsense peptide (CRDGS) the value was 245% (p<0.05). This result is consistent with that of Burdick and Anseth, who reported that RGD in PEG hydrogels failed to promote the proliferation of osteoblasts, and the cell density decreased significantly following in vitro culture for one and two weeks.

### EXAMPLE 3

To investigate the influence of fibronectin peptide fragments (e.g. an RGD-containing peptide) on the fibrous tissue formation in vivo, a gelling suspension of NIH 3T3 fibroblasts in an HA-DTPH/CRGDS-coupled PEGDA mixture (50×10⁶ cell/ml) was injected subcutaneously into the flanks of nude mice. Under these conditions, the gelation occurred in situ, and the hydrogel formed in vivo. Hydrogels without peptide and without cells were used as controls. No signs of biological incompatibility, e.g., necrosis or damage to the surrounding tissues, were observed (results not shown). The hydrogels formed in vivo with no cells present were partially degraded 4 weeks post-injection and at 8 weeks they completely disappeared (data not shown). No tissues formed in these cell-free hydrogels. On the other hand, the hydrogel that had been pre-seeded with cells became more opalescent and elastic with time. At 4 weeks post-injection, immunohistochemical analysis showed procollagen-positive staining, indicating that abundant procollagen production by the encapsulated fibroblasts in gels without and with RGD peptide (data not shown). More uniform fibrous tissue and procollagen was observed found in the crosslinked CRGDS-containing hydrogels than in the hydrogel lacking the peptide (data not shown). At 8 weeks post-injection, uniform fibrous tissue had formed in hydrogels both with and without the cell-adhesive RGDS peptide.

### EXAMPLE 4

As noted previously, in one embodiment, the present invention contemplates attaching one or more domains of native human fibronectin to a derivatized PEG linker to form a first conjugate, followed by attachment of the first conjugate to HA (e.g. derivatized HA). In a preferred embodiment, three functional domains are employed. For example, the present invention contemplates attaching the RGD cell binding site (FNIII₍₈₋₁₁₎, the heparin II binding site (FNIII₍₁₂₋₁₄₎ or FNIII₍₁₂₋₁₅₎) and binding sites for the integrin (IIICS) (see FIG. 1) to a PEG derivative (e.g. PEG-divinylsulfone) to make a first conjugate.

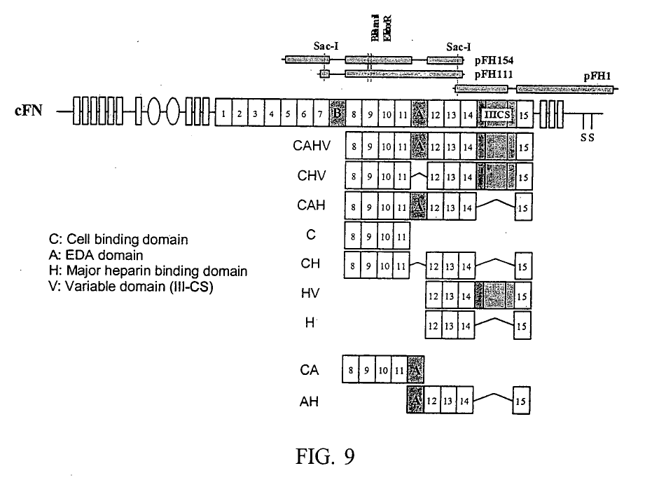

These domains can be prepared recombinantly. Briefly, functional human FN domains (see FIG. 9) have been cloned by PCR using the human cDNA clones pFH1, pFH111 and pFH154, as template or by subcloning of the restriction enzyme fragments from these plasmids. The pFH111 and pFH154 were purchased from ATCC, while the pFH1 clone was obtained form the Japan Health Sciences Foundation. A bacteria expression vector, pETCH, has been constructed by modifying the pET vector from Stratagene. The inserts were cloned at the BamHI and Hind-III sites, and confirmed by DNA sequencing to rule out possible synthesis errors during PCR.

Protein induction and purification procedures have been optimized for each of the FN fragments. Protein expression was induced in the BL21DE3LysS strain of E coli by the addition of 0.5 mM IPTG to the L-Broth and affinity-purified using the Ni-NTA agarose (Qiagen) according to the manufacturer's protocol. After elution with 250 mM imidazole, the protein solution was purified in a G25 gel filtration column equilibrated in PBS, and the aliquots stored at −70° C.

The relevant and available clones are listed in the FIG. 9, which depicts the so-called embryonic or cellular form of FN (cFN). Note that unlike FIG. 1, which depicts plasma FN, the extra domain splice-variants EDA and EDB are present in FIG. 9. The FN clones produced include the functional domains: CAHV, CHV, CAH, C, CH, CV and H, some of which have EDA. The recombinant FN functional domains have three extra amino acids (MetGlySer) at the N-terminus and seven to eight extra amino acids (Thr-Ser-His-His-His-His-His-His-Cys) at the C-terminus (Thr is naturally present at the end of type III repeat 11 and EDA). CAH clone is used as a template for constructing CA and AH (FIG. 9). Oligonucleotides are designed and synthesized to cover the 5′-end and the 3′-end of the EDA domain and include necessary cloning sites. The PCR products are purified, cut with restriction enzymes. The restriction fragments are separated by gel electrophoresis, purified, ligated into the vector, and transformed into competent bacteria DH5a. The clones are confirmed by DNA sequencing, and transformed into BL21DE3-LysS bacteria for protein purification.

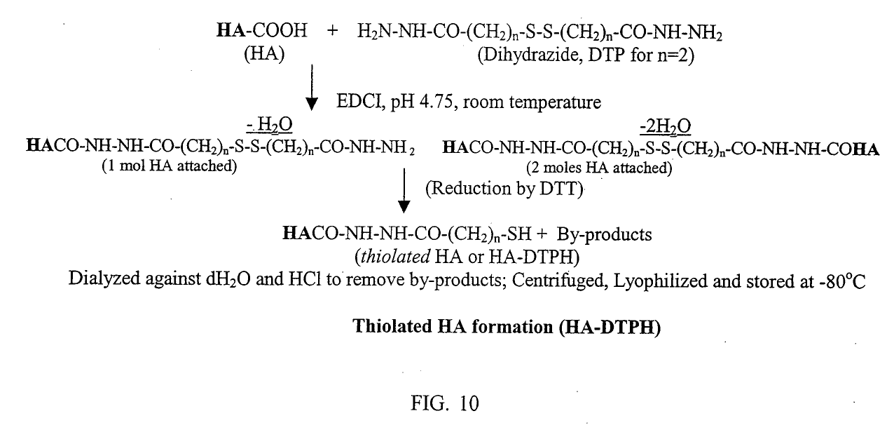

Fermentation-derived hyaluronan (HA, sodium salt, M_{w} 1.5 MDa) was provided by Clear Solutions Biotechnology, Inc. (Stony Brook, N.Y.) and converted to low molecular weight HA (LMW-HA) by acid degradation. Next, dithiobis(propionic dihydrazide) (DTP) was synthesized from the diacid 3,3′-dithiobis (propanoic acid) (Aldrich Chemical co., Milwaukee, Wis.). Next, 1-Ethyl-3-[3-(dimethylamino) propyl]carbodiimide (EDCI) was used to conjugate disulfide containing dihydrazide to the carboxyl terminus of LMW-HA (FIG. 10). The reaction between the carboxylic acid groups of HA with EDCI occurs when the carboxylate is protonated, but for coupling to an amine or hydrazide the pH must be such that the carboxylate is unprotonated and nucleophilic. For HA, the optimal pH to balance these requirements occurs at 4.75. The disulfide that crosslinked HA was reduced by dithiotheritol (DTT) to obtain HA with free thiol groups. Purity and molecular distribution of the thiolated HA was measured by gel permeation chromatography (GPC), which showed a single peak indicating that the HA-DTPH product was free from large and small impurities.

Proton NMR spectral data were obtained using a Varian INOVA 400 at 400 Hz and confirmed the presence of two methylene groups arising from the addition of dihydrazides to the carboxy terminus of HA. The degree of substitution was primarily controlled by the molar ratios of HA, DTP and EDCI, coupled with the reaction time allowed. In a typical reaction 42% substitution was obtained in the final HA-DTPH product indicating that 42% of the glucuronate residues/mole of HA had been converted to thiol-containing derivatives.

At this point, the cysteine-tagged recombinant FN domain(s) could be coupled to the α, β unsaturated ester of PEGDA. Poly(ethylene glycol) diacrylate (PEGDA 3400 and 1000) was synthesized from Poly(ethylene glycol) (Mw 3400 and 1000 Da respectively) according to literature. Degree of substitution: PEGDA 3400, 95%; PEGDA 1000 93%. PEGDA with the appropriate rFNcys domain(s) were dissolved in 5 ml DPBS solution, and stirred for 4 h. In the above stock solutions the molar ratio of peptide to PEGDA was 1/10 and rFNcys domain to PEGDA was 1/200. PEGDA solutions with lower peptide or rFNcys domain concentrations were prepared by diluting the stock solution with blank PEGDA solution. Solutions were sterilized by filtering through 0.22 μm filter. In this chemical reaction, the electron-deficient double bonds of the PEGDA react rapidly with thiols by conjugate addition to give thioether adducts. For the unsubstituted acrylates, the reaction occurs within 5-20 minutes at pH 7.4, room temperature. Reaction with the methacrylate, acrylamide, and methacrylamide derivatives of PEG occur approximately 10×, 100×, and 1000× more slowly, respectively. The density of cys-tagged FN domains coupled to HA-DTPH via PEGDA is readily controlled by varying the molar ratio of cys-FN to PEGDA, which is always in large excess relative to the cys-FN fragment. This permits direct use of the mixture of homobifunctional PEGDA crosslinker with the minor amount of monofunctional FN-cys-PEGDA to crosslink the HA-DTPH solution. It is also important to note that only 50% of the theoretical amount of PEGDA relative to available HA-thiol groups is used; this assures complete consumption of the chemically reactive and potentially toxic acrylate groups, leading to a fully biocompatible crosslinked gel with net free thiols remaining.

At this point, the PEGDA-cysFN domain(s) conjugate can be crosslinked to thiolated HA. HA-DTPH (M_{w}—158 kDa, Mₙ—78 kDa, Polydispersity Index—2.03) was dissolved in serum-free DMEM/F-12 medium or complete DMEM/F-12 medium supplemented with 10% newborn calf serum (for NIH 3T3 fibroblast) or fetal bovine serum (for adult human fibroblast strains) and 50 μg/ml ascorbic acid to give a 1.25% (w/v) solution. pH was adjusted to 7.4 by adding 1.0 N NaOH. Solutions were sterilized through 0.22 μm filter. PEGDA solutions with different concentrations rFNcys functional domains were added into HA-DTPH solution (the ratio of thiols to acrylate was about 2/1), and mixed for 30 seconds. The solution was then injected into each well of 24-well plates. Usually solutions gelled in approximately 7 min. After 1 hour, hydrogels were used directly for experiments or dried in the hood for 2 days to give films.

Michael-type addition of thiols to PEGDA/PEGDA-rFNcys is an ideal reaction for in situ gelation due to its fast reactivity and greater degree of completion, ease of preparation, absence of any by-products and negligible influence of competing reactions with other nucleophiles (viz. amines).

HA-PEDGA, like other hydrogels, is extremely hydrophilic, polyanionic surfaces, preventing cell attachment and limiting their utility for cell growth and tissue remodeling. However, this property has been used to our advantage. Usually it is very difficult to control the surface properties of most artificial materials due to nonspecific protein adsorption onto the surface of the materials. Since hydrogels based on the thiolated HA are largely resistant to protein adsorption, this is not a problem. Rather than nonspecific adsorption, the present invention contemplates specific incorporation of peptides or domains that promote cell attachment, migration and proliferation. This feature selectively enhances the utility of HA-based hydrogels for tissue repair and regeneration.

In experiments using recombinant, cys-tagged FN type III repeats 8-11 (rFNIII₍₈₋₁₁₎Hist₆Cys), which include both the synergy PHSRN and RGD cell binding sites, to decorate HA-PEGA (rFNIII₍₈₋₁₁₎Hist₆Cys-HA-PEGA), CF-31 cells spread by 6 hours on rFNIII₍₈₋₁₁₎Hist₆Cys-HA-PEGDA.

### EXAMPLE 5

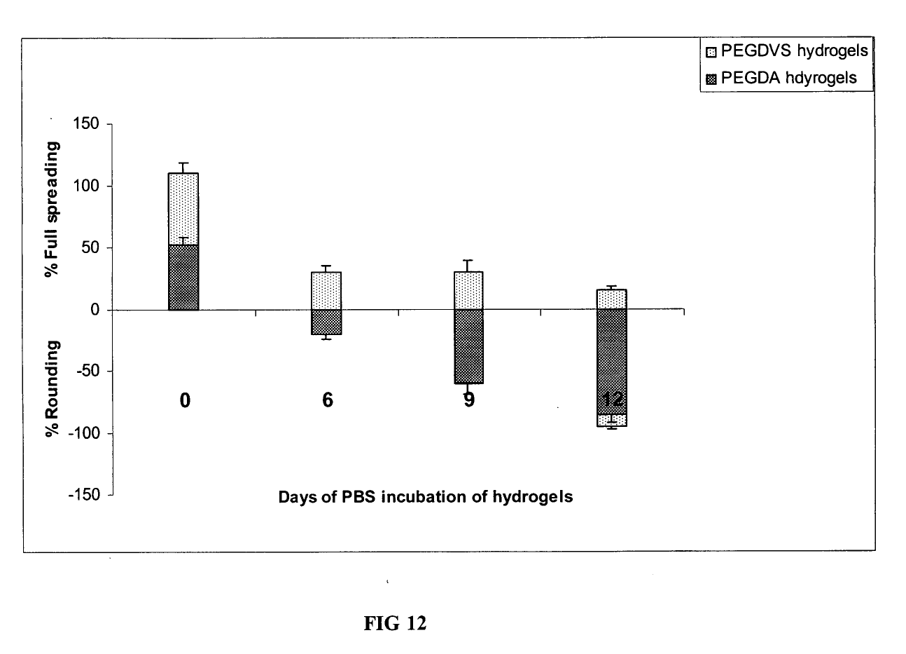

The invention is not limited to any single type of linker for the complex conjugate of hyaluronate and functional fibronectin domains of the invention. Although PEG-divinyl sulfone, PEG-diacrylamide and PEG-diacrylate have been mentioned, the artisan will select linkers that tend to optimize desired properties of the conjugate, one of which is stability in a hydrolytic environment. In an experiment to determine the relative susceptibility of PEGDA-based hydrogels and PEGDVS-based hydrogels to hydrolytic degradation, the two types of hydrogel were incubated for various periods of time under hydrolytic conditions and then “challenged” to support fibroblasts in a morphology (“fully spread”) that is consistent with growth and normal function. From the outset, as FIG. 12 demonstrates, PEGDVS hydrogels supported the “spread-cell” morphology of fibroblasts disposed on the hydrogel surface more robustly than do PEGDA hydrogels. About 25% of cells seeded onto PEGDA hydrogels that have hydrolyzed for 6 days assumed a rounded morphology and none exhibited a fully spread morphology. In contrast, a significant proportion of cells seeded onto PEGDVS hydrogels adhered to the surface in fully spread form even in the case of hydrogel that had hydrolyzed for 12 days.

### EXAMPLE 6

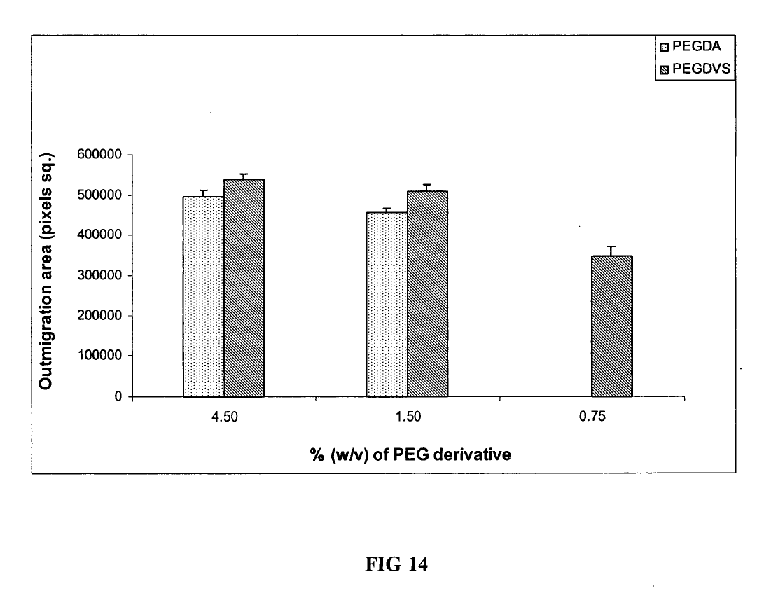

Fibroblasts plated on a hydrogel surface that supports normal cell morphology should also tend to exhibit normal cell physiology. Migration is a hallmark of fibroblast function. Indeed, as previously noted, fibroblast migration is probably more important in wound-healing than fibroblast proliferation. To evaluate the contribution that linker-type makes to fibroblast physiology in the context of the hyaluronate and fibronectin-based conjugate that comprises the matrix of the invention, two species of hydrogel were prepared and subjected to the out-migration assay described above. The results, presented graphically in FIG. 14, demonstrate that PEGDVS is superior to PEGDA in this assay. As the concentration of the PEG derivative was reduced experimentally, the ability of the hydrogel to support out-migration from a start-site (cells in an agarose droplet) declined. In the case of PEGDA, however, the ability simply collapsed at a concentration of 0.75% (w/v). This result is not intended to imply that PEGDVS is the best conceivable linker to select in practicing the invention. The result does show that the out-migration assay is a useful means of evaluating candidate linkers.

### EXAMPLE 7

To evaluate their healing potential in vivo, we injected various formulations (listed below) of our engineered ECM (engECM) constructs into 8 mm punch biopsy wounds created in Yorkshire pigs. A previously reported “re-injury” model was used where the fresh wounds were first allowed to heal spontaneously for 5 days. Thereafter, the wounds were cleansed by curetting the granulation tissue, following which they were filled with the various engECM constructs.

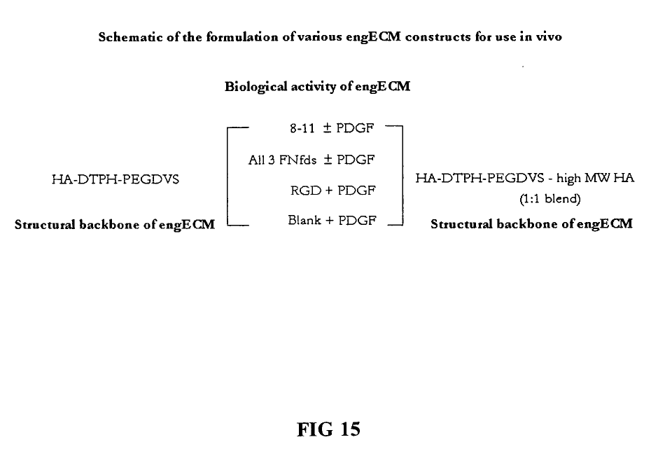

We classified our engECM formulations under two primary categories; first, where we altered the nature of the “structural backbone” and second, where we altered the nature of the “biological activity” (the terms are set off in quotation marks to indicate that they are used herein simply as a means of distinguishing the part of the material comprising peptides or proteins from the part of the material that holds the peptides or proteins). The classification is represented diagrammatically in FIG. 15.

Different structural backbones were introduced by using either the typical crosslinked HA-DTPH-PEGDVS hydrogels or, a 1:1 (volumetric ratio) blend of crosslinked HA-DTPH-PEGDVS hydrogel with high molecular weight HA (MW 1.5 MDa). First, a 1.25% (w/v) solution of HA-DTPH in serum-free DMEM, a 4.5% (w/v) solution of PEGDVS in dPBS (±FN functional domains {or, RGD}±PDGF) and a 1% (w/v) solution of high MW HA in serum-free DMEM were prepared and pH adjusted to 7.40. The HA-DTPH and PEGDVS solutions were sterilized using a 0.22 μm filter, while the sterility of the high MW HA solution was assured by handling the sterile HA powder only in a sterile tissue culture hood. To prepare the typical HA-DTPH-PEGDVS hydrogels, 4 volumes of HA-DTPH solution were added to 1 volume of PEGDVS solution and mixed in a sterile 15 ml conical tube for 30 sec. To prepare the 1:1 blends with high MW HA, equal volumes of the pre-gelled HA-DTPH-PEGDVS solutions were mixed with the high MW HA for approximately 30 sec. Using sterile micropipette and tips, 100 μL of the pre-gelled solutions was then injected into the cleansed wounds. Typical HA-DTPH-PEGDVS solutions gelled in less than 10 minutes, while the 1:1 blends with HMW HA took about 25 minutes to gel.

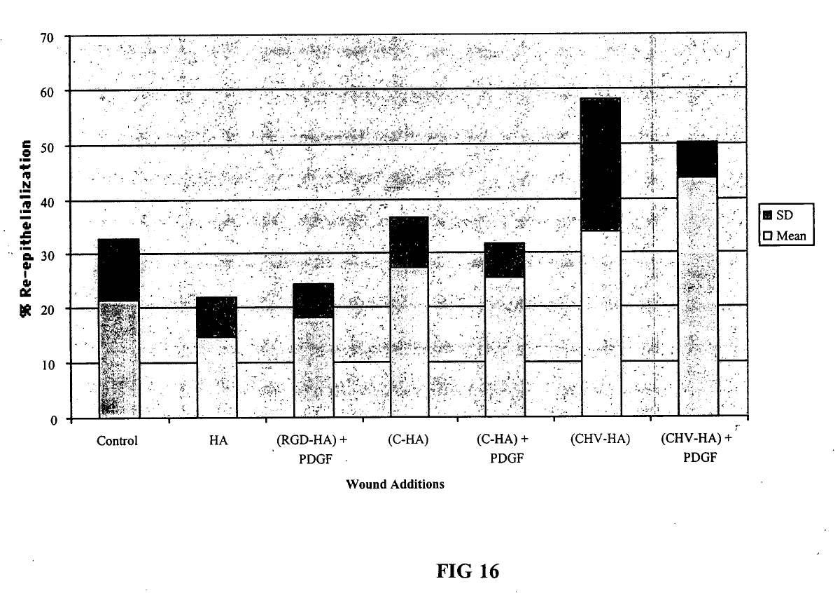

Different biological activities were introduced by using a) the recombinant 8-11 FN functional domain (FNfd) alone, b) a combination of all three recombinant FNfds (viz., 8-11, 12-15 and 12-15V), c) the synthetically derived RGD tri-peptide sequence alone, and d) neither FNfd nor RGD (blank). All the FN functional domains and the RGD peptide were used at an individual bulk density of 0.52 μM (equivalent to a concentration of 1/5000 when expressed as “moles FN functional domains or RGD/moles PEGDVS). A further variation in biological activity was introduced by either adding or not adding PDGF (at 30 ng/ml final concentration) to the FNfd containing PEGDVS solutions. However, the RGD-containing and blank engECM constructs always contained PDGF. These various PEGDVS mixes were then used to prepare the various hydrogel constructs (as described in the previous section). Also, we maintained 6 control samples where engECM constructs were not added at all. The results are summarized graphically in FIG. 16.

From the above, it should be evident that the present invention provides methods and compositions for enhancing and promoting wound healing. Thereafter, such agents can be modified or derivatized and used therapeutically by application directly on wounds. In one embodiment, such constructs are attached to solid supports (e.g. dressings and the like) for application on wounds.

1. A composition, comprising a peptide, said peptide comprising at least three contiguous amino acids from native human fibronectin, covalently attached to a linker, said linker selected from the group consisting of polyethylene glycol and polyethylene glycol derivatives, said linker covalently attached to hyaluronic acid.

2. The composition of claim 1, wherein said linker comprises a polyethylene glycol derivative selected from the group consisting of PEG-divinylsulfone, PEG-diacrylamide and PEG-diacrylate.

3. The composition of claim 1, wherein said peptide has the general formula: X₁RGDX₂ wherein X₁ represents between 0 and 100 additional amino acids, and x₂ of between 0 and 100.

4. The composition of claim 1, wherein said peptide has the general formula:
X₁PHSRNX₂ wherein X₁ represents between 0 and 100 additional amino acids, and X₂ of between 0 and 100.

5. The composition of claim 1, wherein said peptide is selected from the group consisting of SEQ ID NOS 1, 2, 3, 4, 5, 6, 7, 8, 9, 10, 11 and 12.

6. The composition of claim 5, wherein said peptide further comprises a terminal cysteine.

7. A method for treating a wound, comprising a) providing: i) the composition of claim 1 and ii) a subject having at least one wound; and b) administering said composition to said subject under conditions such that the healing of said wound is promoted.

8. The method of claim 7, wherein said linker comprises a polyethylene glycol derivative selected from the group consisting of PEG-divinylsulfone, PEG-diacrylamide and PEG-diacrylate.

9. The method of claim 7, wherein said peptide has the general formula: X₁RGDX₂ wherein X₁ represents between 0 and 100 additional amino acids, and X₂ of between 0 and 100.

10. The method of claim 7, wherein said peptide has the general formula: X₁PHSRNX2 wherein X₁ represents between 0 and 100 additional amino acids, and X₂ of between 0 and 100.

11. The method of claim 7, wherein said peptide is selected from the group consisting of SEQ ID NOS 1, 2, 3, 4, 5, 6, 7, 8, 9, 10, 11 and 12.

12. The method of claim 7, wherein said peptide comprises the amino acid sequence CRGD.

13. The method of claim 7, wherein said subject is a diabetic.

14. The method of claim 13, wherein said wound is a chronic wound.

15. The method of claim 7, wherein said wound is a surgical wound.

16. A composition, comprising at least two domains from native human fibronectin, covalently attached to a linker, said linker selected from the group consisting of polyethylene glycol and polyethylene glycol derivatives, said linker covalently attached to hyaluronic acid.

17. The composition of claim 16, wherein said linker comprises a polyethylene glycol derivative selected from the group consisting of PEG-divinylsulfone, PEG-diacrylamide and PEG-diacrylate.

18. The composition of claim 16, wherein said domains are non-contiguous.

19. The composition of claim 16, wherein three domains from native human fibronectin are covalently attached to said linker.

20. The composition of claim 19, wherein said three domains are the RGD cell binding site, a heparin II binding site and a binding site for the integrin

21. The composition of claim 20, wherein each of said three domains are modified by the addition of cysteine prior to covalently attaching said domains to said linker.

22. The composition of claim 16, wherein at least one of said domains comprises an amino acid sequence selected from the group consisting of SEQ ID NOS 1, 2, 3, 4, 5, 6, 7, 8, 9, 10, 11 and 12.

23. A method for treating a wound, comprising a) providing: i) the composition of claim 16 and ii) a subject having at least one wound; and b) administering said composition to said subject under conditions such that the healing of said wound is promoted.

24. The method of claim 22, wherein said wound is a burn.

25. The method of claim 22, wherein said subject is a diabetic.

26. The method of claim 24, wherein said wound is a chronic wound.

27. The method of claim 22, wherein said wound is a surgical wound.

28. A method, comprising a) providing a fibronectin peptide fragment, a polyethylene glycol derivative selected from the group consisting of PEG-divinylsulfone, PEG-diacrylamide and PEG-diacrylate, and hyaluronic acid; b) covalently attaching said fibronectin peptide fragment to said polyethylene glycol derivative to create a first conjugate; c) reacting said first conjugate with said hyaluronic acid to create a second conjugate.

29. A method, comprising a) providing at least two domains of native human fibronectin, a polyethylene glycol derivative selected from the group consisting of PEG-divinylsulfone, PEG-diacrylamide and PEG-diacrylate, and hyaluronic acid; b) covalently attaching said domains to said polyethylene glycol derivative to create a first conjugate; c) reacting said first conjugate with said hyaluronic acid to create a second conjugate.
